(19) United States
(12) Patent Application Publication
(10) Pub. No.: US 2011/0086388 A1
(43) Pub. Date: Apr. 14, 2011

(54) CHAPERONE-ASSISTED PROTEIN EXPRESSION AND METHODS OF USE

(75) Inventors: Dewey G. McCafferty, Chapel Hill, NC (US); Amanda J. Hoertz, Morrisville, NC (US)

(73) Assignee: Duke University

(21) Appl. No.: 12/888,061

(22) Filed: Sep. 22, 2010

Related U.S. Application Data
(60) Provisional application No. 61/244,498, filed on Sep. 22, 2009.

(51) Int. Cl. C12P 21/00; C07K 14/195; C12N 9/00

(52) U.S. Cl. 435/69.1; 530/350; 435/183

(57) ABSTRACT

The present disclosure provides methods of utilizing chaperone proteins for the production of active protein such as those encoded by the genes of natural biosynthetic clusters. The methods provided herein have applicability for a wide variety of genes ranging from small fatty acid biosynthetic genes to large non-ribosomal peptide synthetase genes.

### RELATED APPLICATIONS

This patent application claims the benefit under 35 U.S.C. §119(e) of U.S. Provisional Application No. 61/244,498, filed Sep. 22, 2009, the disclosure of which is incorporated herein by reference in its entirety.

### STATEMENT OF GOVERNMENT SUPPORT

This invention was produced in part using federal funds under NIH Grant No. NIH RO1 AI46611. Accordingly, the U.S. Government has certain rights in this invention.

### FIELD OF THE DISCLOSURE

The present disclosure relates generally to the field of recombinant DNA technology. Specifically, the present disclosure relates to the use of chaperone proteins and other proteins similar to them to obtain enzymatically active proteins that can be manipulated for applications such as high throughput screening or combinatorial biosynthesis.

### BACKGROUND OF THE DISCLOSURE

The biosynthetic clusters of natural products have been extensively explored as potential new drug discovery leads. Polyketide (PKS) and non-ribosomal peptide synthetases (NRPS) have been especially attractive due to their modular nature that suggests these enzymes are promising candidates for combinatorial biosynthesis and directed engineering. Although some enzymes have been successfully expressed in heterologous hosts, most present a significant challenge to clone and express.

Production of proteins of interest in native systems is full of complications, including inability to produce high concentrations of protein, varying degrees of difficulty involving the growth of the host organism, and inability to preferentially purify the desired protein. Therefore, it would be useful to devise a method for the expression and isolation of increased amounts of soluble, active protein from genes of natural biosynthetic clusters. Moreover, it would be beneficial for such methods to work on a wide variety of genes ranging from small fatty acid biosynthetic genes to large, non-ribosomal peptide synthetases.

### SUMMARY OF THE DISCLOSURE

The present disclosure provides methods which allow for a high percentage of success in producing soluble proteins of interest and the broad applicability of chaperone proteins to natural biosynthetic clusters. Moreover, the methods provided herein enable detailed investigations into substrate specificity, function and mechanisms of action of important novel enzymes, thereby enabling combinatorial biosynthesis to make, as an example, unique or modified antibiotics and therapeutics. Furthermore, the methods of the present disclosure can be used for a wide variety of genes ranging from small fatty acid biosynthetic genes to large non-ribosomal peptide synthetase genes.

One aspect of the present disclosure provides a method for the soluble expression of a protein of interest produced from a gene or nucleotide sequence of interest comprising, consisting of, or consisting essentially of co-expressing the nucleotide sequence of interest with at least one nucleotide sequence encoding at least one chaperone protein (e.g., a chaperonin protein) in an expression system, and collecting said solubly expressed protein of interest.

In certain embodiments, the nucleotide sequence of interest and at least one nucleotide sequence encoding a chaperone protein are on the same plasmid. In other embodiments, they are on different plasmids. In some embodiments, the plasmid comprises, consists essentially of or consists of the pLAC1 plasmid. In certain embodiments, the expression system comprises the Streptomyces lividans expression system and/or chaperone proteins.

In other embodiments, the at least one nucleotide sequence encoding at least one chaperone protein comprises the GroESL chaperone system. In preferred embodiments, the GroESL chaperone system comprises one or more of the chaperone proteins GroES, GroEL1 and GroEL2.

In yet another embodiment, the nucleotide sequence encoding the protein of interest is in the form of a biosynthetic cluster.

In other embodiments, the nucleotide sequence of interest encodes a protein involved in the biosynthesis of an antibiotic. In certain embodiments, the antibiotic is a lipopeptide antibiotic. In such embodiments, lipopeptide antibiotic is selected from the group consisting of surfactin, ramoplanin, daptomycin and mycosubtilin.

Compositions comprising proteins of interest as described herein are also provided.

These and other novel features and advantages of the disclosure will be fully understood from the following detailed description and the accompanying drawings.

### BRIEF DESCRIPTION OF THE DRAWINGS

FIG. 1 is a depiction of the structures of enduracidin A and ramoplanin A2 (Yin, X. et al., (2006) Microbiology 152:2969-2983). The fatty acid attached at the N-terminus of ramoplanin A2 is highlighted.

FIG. 2 shows a Ramo17 ATP-PPi exchange assay. Shown are two replicate purifications, each performed in triplicate where the amino acid incubated with the enzyme was compared against a control of no amino acid. Data was normalized to the most active amino acid.

FIGS. 3A-C show the results of the Ramo11—Acyl carrier protein. FIG. 3A is a depiction of the chemical reaction of co-enzyme A by Sfp for use in BODIPY-CoA assay. FIG. 3B shows the results of a BODIPY-CoA assay using clone Ramo11. FIG. 3C is a gel electrophoresis showing the size of the Ramo11 clone.

FIGS. 4A-C show the results of VbsS—Viomycin non-ribosomal peptide synthetase. FIG. 4A depicts the chemical reaction of coenzyme A by Sfp with VbsS for use in BODIPY-CoA assay. FIG. 4B is a gel electrophoresis showing the VbsS DNA. FIG. 4C is a western blot showing the VbsS and Sfp proteins.

FIG. 5 is a map of the plasmid pLacI-GroESEL.

FIG. 6 shows a schematic of the NRPS and FA biosynthetic enzymes composing the cluster. Genes implicated in fatty acid biosynthesis are colored in dark grey; genes associated with non-ribosomal peptide synthesis shown in light grey; all other genes in the cluster are shown in black.

FIG. 7 shows HPLC analysis of Ramo11 incubated in the absence ( - - - ) and presence (-) of the phosphopantetheinyl transferase enzyme. Peaks corresponding to the retention time of the holo-form (t_{R}=41.3) and apo-form (t_{R}=44.5 min) were collected and subjected to MALDI analysis.

FIG. 8 is a kinetic profile of Ramo16 (10 μM) with varied acetoacetyl-CoA (0 to 5 mM) and fixed NADH (250 μM).

### DETAILED DESCRIPTION

Unless otherwise defined, all technical terms used herein have the same meaning as commonly understood by one of ordinary skill in the art to which this disclosure belongs.

The articles “a” and “an” and “the” are used herein to refer to one or to more than one (i.e. at least one) of the grammatical object of the article. By way of example, “an element” means at least one element and can include more than one element.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a compound or agent of this invention, dose, time, temperature, and the like, is meant to encompass variations of ±20%, ±10%, ±5%, ±1%, ±0.5%, or even ±0.1% of the specified amount.

### DEFINITIONS

The abbreviations in the specification correspond to units of measure, techniques, properties or compounds as follows: “min” means minutes, “h” means hour(s), “ul” means microliter(s), “ml” means milliliter(s), “mM” means millimolar, “M” means molar, “mmole” means millimole(s), “kb” means kilobase, “bp” means base pair(s), and “IU” means International Units. “Polymerase chain reaction” is abbreviated PCR; “Reverse transcriptase polymerase chain reaction” is abbreviated RT-PCR; “DNA binding domain” is abbreviated DBD; “Ligand binding domain” is abbreviated LBD; “Untranslated region” is abbreviated UTR; and “Sodium dodecyl sulfate” is abbreviated SDS.

As used herein, the term “gene of interest” or “nucleotide sequence of interest” refers to the gene or nucleotide sequence which encodes a protein that is desired to be isolated (e.g., a protein of interest). In some embodiments, the gene of interest or nucleotide sequence of interest is one which is part of a biosynthetic cluster. The gene of interest or nucleotide sequence of interest will be dependent on a number of factors that can be readily detei mined by one skilled in the art. In certain embodiments of the present disclosure, the gene of interest or nucleotide sequence of interest encodes an enzyme involved in the synthesis of an antibiotic, such as a lipopeptide antibiotic. Such antibiotics include, but are not limited to, ramoplanin, surfactin, mycosubtilin, and daptomycin. In preferred embodiments, the antibiotic is ramoplanin.

As used herein, the term “chaperone protein” refers to those proteins that assist in the non-covalent folding/unfolding and assembly/disassembly of other macromolecular structures, but do not occur in these structures when the latter are performing their normal biological functions. Examples of chaperone proteins include, but are not limited to, histones, GroESL chaperone system (GroES, GroEL1 and GroEL2), heat shock proteins, BiP, GRP94, GRP170, calnexin, calreticulin, HSP47, ERp29, protein disulfide isomerase (PDI), peptidyl prolyl cis-trans-isomerase (PPI) and ERp57.

Proteins of Interest

Proteins that may be expressed using the methods disclosed herein include any protein of interest, including proteins of interest for which increased expression yield, improvement of protein folding and/or functionality is desired. In some embodiments, the protein is an enzyme or a portion thereof. In some embodiments, expression results in the protein of interest being soluble. “Soluble” as used herein refers to the protein remaining in solution in the cell (or media if excreted), often the result of proper folding of the nascent protein, as opposed to insoluble (which may form “inclusion bodies”). Thus, for example, when a host cell expressing the protein of interest is lysed, the expressed protein of interest is easily collected from the cytosolic fraction.

Examples of proteins of interest that may be expressed using the methods disclosed herein include, but are not limited to, non-ribosomal peptide synthases (NRPSs). A NRPS gene or nucleic acid encoding one or more domains of a NRPS may be provided for use in the expression systems as disclosed herein. The term “NRPS gene” or refers to one or more genes or nucleic acids encoding NRPSs for producing functional secondary metabolites when under the direction of one or more compatible control elements. These genes are normally found on a “biosythetic cluster” in the genome.

As known in the art, “non-ribosomal peptides” are a class of peptide secondary metabolites produced mainly by microorganisms such as bacteria and fungi. Non-ribosomal peptides are typically synthesized by non-ribosomal peptide synthetases (NRPS). Known non-ribosomal peptides include, but are not limited to, antibiotics, cytostatics, immunosuppressants, toxins, siderophores and pigments. Also contemplated is the synthesis of precursors of these peptides, which may be useful in their subsequent synthesis, as well as derivatives of these peptides (e.g., 7-aminoactinomycin D (7-AAD) as a fluorescent derivative of actinomycin).

Examples of antibiotics include, but are not limited to, actinomycin (e.g., actinomycin D), bacitracin, daptomycin, vancomycin, tyrocidine, gramicidin, thiostrepton, and zwittermicin A. ACV tripeptide is an example of an antibiotic precursor.

Further non-limiting examples of antibiotics include sulfa drugs (e.g., sulfanilamide, sulfamethoxazole); folic acid analogs (e.g., trimethoprim); beta-lactams; penicillins (e.g., ampicillin, amoxicillin, penicilin G); cephalosporins (e.g., cephalexin, cefaclor, cefixime); carbapenams (e.g., meropenem, ertapenem); aminoglycosides (e.g., streptomycin, kanamycin, neomycin, gentamycin); tetracyclines (e.g., chlorotetracycline, oxytetracyclin, doxycycline); macrolides (e.g., erythromycin, clarithromycin); lincosamides (e.g., clindamycin); streptogramins (e.g., quinupristin, dalfopristin); fluoroquinolones (e.g., ciprofloxacin, levofloxacin, and norfloxacin); polypeptides (e.g., polymixins); rifampin; mupirocin; cycloserine; aminocyclitol (e.g., spectinomycin); glycopeptides (e.g., vancomycin); oxazolidinones (e.g., linezoid); lipopeptides (e.g., daptomycin, ramoplanin, enduracidin, surfactin, mycosubtilin). See, e.g., Antibiotics: Actions, Origin, Resistance, by Christopher Walsh, ASM Press, 2003 pp 1-340).

Examples of cytostatics include, but are not limited to, epothilone and bleomycin. Examples of immunosuppressants include, but are not limited to, ciclosporine (e.g., cyclosporine A). Examples of siderophores include, but are not limited to, enterobactin and myxochelin A. Examples of pigments include, but are not limited to, indogoidine. Examples of toxins include, but are not limited to, microcystins, nodularins (cyanotoxins from cyanobacteria), HC-toxin, AM-toxin and victorin. Examples of nitrogen storage polymers include, but are not limited to, cyanophycin.

The NRPS enzymes are generally composed of modules where a minimal module contains three domains, an adenylation domain, a thiolation domain, and a condensation domain.

The adenylation domain is typically about 60 kDa. The main function of this domain is to select and activate a specific amino acid as an aminoacyl adenylate. Based on its function, the adenylation domain regulates the sequence of the peptide being produced. Once charged (as an amino acyl adenylate moiety), the amino acid is transferred to a thiolation domain (peptidyl carrying center).

The second domain is the thiolation domain, also referred to as a peptidyl carrier protein. This domain is typically 8-10 kDa and contains a serine residue that is post-translationally modified with a 4-phosphopantetheine group. This group acts as an acceptor for the aminoacyl adenylate moiety on the amino acid. A nucleophilic reaction leads to the release of the aminoacyl adenylate and conjugation of the amino acid to thiolation domain via a thioester bond.

The third domain is the condensation domain. This domain is typically about 50-60 kDa in size. The main function of this domain is to catalyze the formation of a peptide bond between two amino acids. In this reaction an upstream tethered peptidyl group is translocated to the downstream aminoacyl-s-Ppant and linked to the amino acid by peptide bond formation.

This minimal module for chain extension is typically repeated within a synthetase. Additionally, and typically, a co-linear relationship exists between the number of modules present and the number of amino acids in the final product with the order of the modules in the synthetase determining the order of the amino acids in the peptide. This 1:1 relationship, with every amino acid in the product having one module within the enzyme, is referred to as the co-linearity rule. Examples have been found that violate this rule, and in such cases, the NRPS contains more modules than one would expect based on the number of amino acids incorporated in the peptide product (Challis et al., (2000) Chem. Biol. 7:211-24). In some cases the minimal module also is supplemented with additional domains (epimerization, N- or C-methylation, or cyclization domain), with their position in the synthetase determining the substrate upon which they can act. In addition, it has been observed that NRPSs contain inter-domain spacers or linker regions. It has been proposed that these spacers may play a critical role in communication between domains, modules, and even entire synthetases.

There are highly conserved motifs in the catalytic domains of peptide synthetases including: 10 conserved motifs in the adenylation domain; 1 conserved motif in the thiolation domain; 7 conserved motifs in the condensation domain; 1 conserved motif in the thioesterase domain; 7 conserved motifs in the epimerization domains; and 3 conserved motifs in the N-methylation domains. These are detailed in Marahiel et al., Chemical Rev. 1997; 97:2651-73. In addition to modifications such as epimerization, methylation and cyclization during peptide synthesis, post-translational modifications including methylation, hydroxylation, oxidative cross-linking and glycosylation can occur (Walsh et al., (2001) Cum Opin. Chem. Biol. 5:525-34).

As used herein, the term “polyketide synthase” or “PKS” refers to the complex of enzymatic activities (domains) responsible for the biosynthesis of polyketides including, for example, ketoreductase, dehydratase, acyl carrier protein, enoylreductase, ketoacyl ACP synthase, and acyltransferase. A functional PKS is one that catalyzes the synthesis of a polyketide. The term “PKS genes” refers to one or more genes encoding various polypeptides useful for producing functional polyketides, e.g., epothilones A and B, when under the direction of one or more compatible control elements.

Nucleotides are indicated by their bases by the following standard abbreviations: adenine (A), cytosine (C), thymine (T), uracil (U), and guanine (G). Amino acids are likewise indicated by the following standard abbreviations: alanine (ala; A), arginine (Arg; R), asparagine (Asn; N), aspartic acid (Asp; D), cysteine (Cys; C), glutamine (Gln; Q), glutamic acid (Glu; E), glycine (Gly; G), histidine (His; H), isoleucine (Ile; I), leucine (Leu; L), lysine (Iys; K), methionine (Met; M), phenylalanine (Phe; F), proline (Pro; P), serine (Ser; S), threonine (Thr; T), tryptophan (Trp; W), tyrosine (Tyr; Y), and valine (Val; V). Furthermore, (Xaa; X) represents any amino acid.

Molecular Biology

In accordance with the present disclosure there may be employed conventional molecular biology, microbiology, and recombinant DNA techniques within the skill of the art. Such techniques are explained fully in the literature (See, e.g., Sambrook, Fritsch & Maniatis, (1989) Molecular Cloning: A Laboratory Manual, Second Edition, Cold Spring Harbor Laboratory Press, Cold Spring Harbor, N.Y. (herein “Sambrook et al., 1989”); DNA Cloning: A Practical Approach, Volumes I and II (1985) D. N. Glover ed.; Oligonucleotide Synthesis (1984) M. J. Gait ed.; Nucleic Acid Hybridization (1985) B. D. Hames & S. J. Higgins eds.; Transcription And Translation (1984) B. D. Hames & S. J. Higgins, eds.; Animal Cell Culture (1986) R. I. Freshney, ed.; Immobilized Cells And Enzymes (1986) IRL Press; B. Perbal, (1984) A Practical Guide To Molecular Cloning; F. M. Ausubel et al. (eds.), (1994) Current Protocols in Molecular Biology, John Wiley & Sons, Inc.

“Amplification” of DNA as used herein denotes the use of polymerase chain reaction (PCR) to increase the concentration of a particular DNA sequence within a mixture of DNA sequences. For a description of PCR see Saiki et al., (1988) Science 239:487.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear (e.g., restriction fragments) or circular DNA molecules, plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation.

A “polynucleotide” or “nucleotide sequence” is a series of nucleotide bases (also called “nucleotides”) in a nucleic acid molecule, such as DNA and RNA, and means any chain of two or more nucleotides. A nucleotide sequence typically carries genetic information, including the information used by cellular machinery to make proteins and enzymes. These terms include double or single stranded genomic and cDNA, RNA, any synthetic and genetically manipulated polynucleotide, and both sense and anti-sense polynucleotide (although only sense stands are being represented herein). This includes single- and double-stranded molecules, i.e., DNA-DNA, DNA-RNA and RNA-RNA hybrids, as well as “protein nucleic acids” (PNA) formed by conjugating bases to an amino acid backbone. This also includes nucleic acids containing modified bases, for example thio-uracil, thio-guanine and fluoro-uracil. As used herein in various embodiments of this invention, the terms “nucleotide sequence” and “gene” are intended to be interchangeable.

Isolated or purified proteins and/or nucleotides are typically removed from cells. The level of purity may be at least 1%, 5%, 10%, 25%, 33%, 50%, 75%, or 90%. Purification of proteins may be achieved by any method known in the art, including but not limited to immunopurification methods, such as immunoaffinity columns. The protein will typically have a sequence which is at least at least 95%, 97%, 98%, or 99% identical to the amino acid sequence known for the protein. The variation in sequence will accommodate different allelic forms of the protein.

The nucleic acid molecules and nucleotide sequences herein may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps,” substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, biotin, and the like.

A “promoter” or “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase. The promoter may be operatively associated with other expression control sequences, including enhancer and repressor sequences.

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

The term “gene,” also called a “structural gene” means a DNA sequence that codes for or corresponds to a particular sequence of amino acids which comprise all or part of one or more proteins (e.g., enzymes), and may or may not include regulatory DNA sequences, such as promoter sequences, which determine for example the conditions under which the gene is expressed. Some genes, which are not structural genes, may be transcribed from DNA to RNA, but are not translated into an amino acid sequence. Other genes may function as regulators of structural genes or as regulators of DNA transcription.

A coding sequence is “under the control of” or “operatively associated with” expression control sequences in a cell when RNA polymerase transcribes the coding sequence into RNA, particularly mRNA, which is then trans-RNA spliced (if it contains introns) and translated into the protein encoded by the coding sequence.

The term “expression control sequence” refers to a promoter and any enhancer or suppression elements that combine to regulate the transcription of a coding sequence. In a preferred embodiment, the element is an origin of replication.

The terms “vector,” “cloning vector” and “expression vector” refer to the vehicle by which DNA can be introduced into a host cell, resulting in expression of the introduced sequence. In one embodiment, vectors comprise a promoter and one or more control elements (e.g., enhancer elements) that are heterologous to the introduced DNA but are recognized and used by the host cell. In another embodiment, the sequence that is introduced into the vector retains its natural promoter that may be recognized and expressed by the host cell (Bormann et al., (1996) J. Bacteriol 178:1216-1218).

An “intergeneric vector” is a vector that permits intergeneric conjugation, i.e., utilizes a system of passing DNA from E. coli to actinomycetes directly (Keiser, T. et al., (2000) Practical Streptomyces Genetics, John Innes Foundation, John Innes Centre (England)). Intergeneric conjugation has fewer manipulations than transformation.

Vectors typically comprise the nucleic acid of a transmissible agent, into which foreign nucleic acid is inserted. A common way to insert one segment of nucleic acid into another segment involves the use of enzymes called restriction enzymes that cleave nucleic acids at specific sites (specific groups of nucleotides) called restriction sites. A “cassette” refers to a nucleic acid coding sequence or segment of the nucleic acid that codes for an expression product that can be inserted into a vector at defined restriction sites. The cassette restriction sites are designed to ensure insertion of the cassette in the proper reading frame. Generally, foreign nucleic acid is inserted at one or more restriction sites of the vector nucleic acid, and then is carried by the vector into a host cell along with the transmissible vector nucleic acid. A segment or sequence of nucleic acid having inserted or added nucleic acids, such as an expression vector, can also be called a “nucleic acid construct.” A common type of vector is a “plasmid,” which in some embodiments is a self-contained molecule of double-stranded DNA, usually of bacterial origin, that can readily accept additional (foreign) DNA and which can be readily introduced into a suitable host cell. A plasmid vector often contains coding and promoter DNA and has one or more restriction sites suitable for inserting foreign DNA. Coding DNA is a DNA sequence that encodes a particular amino acid sequence for a particular protein or enzyme. Promoter DNA is a DNA sequence which initiates, regulates, or otherwise mediates or controls the expression of the coding DNA. Promoter DNA and coding DNA may be from the same gene or from different genes, and may be from the same or different organisms. Recombinant cloning vectors will often include one or more replication systems for cloning or expression, one or more markers for selection in the host, e.g. antibiotic resistance, and one or more expression cassettes. Vector constructs may be produced using conventional molecular biology and recombinant DNA techniques within the skill of the art. Such techniques are explained fully in the literature (see, e.g., Sambrook, et al., 1989; Glover, D. N. ed., (1985) DNA Cloning. A Practical Approach, Volumes I and II; F. M. Ausubel et al. (eds.), (1994) Current Protocols in Molecular Biology, John Wiley & Sons, Inc.

As used herein, the term “cosmid” refers to DNA from a bacterial virus into which is spliced a small fragment of a genome to be amplified and sequenced. Typically, a cosmid contains the cos gene of phage lambda and can be packaged in a lambda phage particle for infection into E. coli, thereby permitting cloning of larger DNA fragments that can be introduced into bacterial hosts in plasmid vectors.

The terms “express” and “expression” mean allowing or causing the information in a gene or nucleotide sequence to become manifest, for example producing a protein by activating the cellular functions involved in transcription and translation of a corresponding gene or nucleotide sequence. A nucleotide sequence is expressed in or by a cell to form an “expression product” or “gene product” such as a protein. The expression product itself, e.g., the resulting protein, may also be said to be “expressed” by the cell or in an expression system. An expression product can be characterized as intracellular, extracellular or secreted. The term “intracellular” means something that is inside a cell. The term “extracellular” means something that is outside a cell. A substance is “secreted” by a cell if it appears in significant measure outside the cell, from somewhere on or inside the cell. The term “transfection” means the introduction of a foreign nucleic acid into a cell.

The term “transformation” means the introduction of a “foreign” (i.e. extrinsic or extracellular) gene, DNA or RNA sequence to a cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences, such as start, stop, promoter, signal, secretion, or other sequences used by a cells genetic machinery. The gene or sequence may include nonfunctional sequences or sequences with no known function. A host cell that receives and expresses introduced DNA or RNA has been “transformed” and is a “transformant” or a “clone.” The DNA or RNA introduced to a host cell can come from any source, including cells of the same genus or species as the host cell, or cells of a different genus or species.

The term “host cell” means any cell of any organism that is selected, modified, transformed, grown or used or manipulated in any way for the production of a substance by the cell. For example, a host cell may be one that is manipulated to express a particular gene, a DNA or RNA sequence, a protein or an enzyme. Host cells can further be used for screening or other assays that are described infra. Host cells may be cultured in vitro or one or more cells in a non-human animal (e.g., a transgenic animal or a transiently transfected animal). For the present invention, host cells include but are not limited to Streptomyces species and E. coli.

The term “expression system” means any suitable expression system. Examples include a host cell and compatible vector under suitable conditions, e.g. for the expression of a protein coded for by foreign (e.g., heterologous) nucleotide sequence carried by the vector and introduced to the host cell. In a specific embodiment, the host cell of the present invention is a Gram-negative or Gram-positive bacterial cell. These bacteria include, but are not limited to, E. coli and Streptomyces species. An example of a Streptomyces species that may be used includes, but is not limited to, Streptomyces coelicolor, Streptomyces lividans, and Streptomyces hygroscopicus. In vitro expression in cell-free extracts (e.g., in vitro expression systems) may also be used, as are well known in the art.

The term “heterologous” refers to a combination of elements not naturally occurring. For example, a heterologous nucleotide sequence or nucleic acid molecule refers to a nucleotide sequence or nucleic acid molecule not naturally located in the cell, or in a chromosomal site of the cell. In some embodiments, the heterologous nucleotide sequence can encode a protein or gene product foreign to the cell into which it has been introduced. For example, the present invention includes chimeric nucleotide molecules that comprise a first nucleotide sequence and a heterologous nucleotide sequence which is not part of the first nucleotide sequence. In this context, the heterologous nucleotide sequence refers to a nucleotide sequence that is not naturally located within another sequence or organism. Alternatively, the heterologous nucleotide sequence may be naturally located within the sequence, but is found at a location where it does not naturally occur. A heterologous expression regulatory element is such an element is operatively associated with a different nucleotide sequence than the one it is operatively associated with in nature. In the context of the present invention, a nucleotide sequence encoding a protein of interest can be heterologous to the vector nucleotide sequence in which it is inserted for cloning or expression, and/or it can be heterologous to a host cell containing such a vector, in which it is expressed.

The terms “mutant” and “mutation” mean any detectable change in genetic material, e.g. nucleotide sequence, or any process, mechanism, or result of such a change. This includes gene mutations, in which the structure (e.g. nucleotide sequence) of a gene is altered, any gene or nucleotide sequence arising from any mutation process, and any expression product (e.g. protein or enzyme) expressed by a modified gene or nucleotide sequence.

The term “variant” may also be used to indicate a modified or altered gene, nucleotide sequence, enzyme, cell, etc., i.e., any kind of mutant. Two specific types of variants are “sequence-conservative variants,” a nucleotide sequence where a change of one or more nucleotides in a given codon position results in no alteration in the amino acid encoded at that position, and “function-conservative variants,” where a given amino acid residue in a protein or enzyme has been changed without altering the overall conformation and function of the polypeptide. Amino acids with similar properties are well known in the art. Amino acids other than those indicated as conserved may differ in a protein or enzyme so that the percent protein or amino acid sequence similarity between any two proteins of similar function may vary and may be, for example, from 70% to 99% as determined according to an alignment scheme such as by the Clustal Method, wherein similarity is based on the algorithms available in MEGALIGN. A “function-conservative variant” also includes a polypeptide or enzyme which has at least 60% amino acid identity as determined by BLAST or FASTA alignments, preferably at least 75%, more preferably at least 85%, and most preferably at least 90%, and which has the same or substantially similar properties or functions as the native or parent protein or enzyme to which it is compared.

As used herein, the terms “homologous” and “homology” refer to the relationship between proteins that possess a “common evolutionary origin,” including proteins from superfamilies (e.g., the immunoglobulin superfamily) and homologous proteins from different species (e.g., myosin light chain, etc.) (Reeck et al., (1987) Cell 50:667, 1987). Such proteins (and their encoding sequences) have sequence homology, as reflected by their sequence similarity, whether in terms of percent similarity or the presence of specific residues or motifs at conserved positions.

Accordingly, the term “sequence similarity” or “identity” refers to the degree of identity or correspondence between nucleic acid or amino acid sequences of proteins that may or may not share a common evolutionary origin (see Reeck et al., supra). However, in common usage and in the instant application, the term “homologous,” when modified with an adverb such as “highly,” may refer to sequence similarity and may or may not relate to a common evolutionary origin.

In certain embodiments, two nucleotide sequences are “substantially homologous” or “substantially similar” or “substantially identical” when at least about 80%, and most preferably at least about 90% or 95% of the nucleotides match over the defined length of the nucleotide sequences, as determined by sequence comparison algorithms, such as BLAST, FASTA, DNA Strider, etc. An example of such a sequence is an allelic or species variant of the specific genes or nucleotide sequences of the invention. Sequences that are substantially homologous can be identified by comparing the sequences using standard software available in sequence data banks, and/or in a Southern hybridization experiment under, for example, stringent conditions as defined for that particular system.

Similarly, in a particular embodiment, two amino acid sequences are “substantially homologous” or “substantially similar” or “substantially identical” when greater than 80% of the amino acids are identical, or greater than about 90% are similar. Preferably, the amino acids are functionally identical. Preferably, the similar or homologous sequences are identified by alignment using, for example, the GCG (Genetics Computer Group, Program Manual for the GCG Package, Version 10, Madison, Wis.) pileup program, or any of the programs described above (BLAST, FASTA, etc.).

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., supra). The conditions of temperature and ionic strength determine the “stringency” of the hybridization. For preliminary screening for homologous nucleic acids, low stringency hybridization conditions, corresponding to a Tₘ (melting temperature) of 55° C., can be used, e.g., 5×SSC, 0.1% SDS, 0.25% milk, and no formamide; or 30% formamide, 5×SSC, 0.5% SDS). Moderate stringency hybridization conditions correspond to a higher Tₘ, e.g., 40% formamide, with 5× or 6×SCC. High stringency hybridization conditions correspond to the highest Tₘ, e.g., 50% formamide, 5× or 6×SCC. SCC is a 0.15M NaCl, 0.015M Na-citrate. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., supra, 9.50-9.51). For hybridization with shorter nucleic acids, i.e., oligonucleotides, the position of mismatches becomes more important, and the length of the oligonucleotide determines its specificity (see Sambrook et al., supra, 11.7-11.8). A minimum length for a hybridizable nucleic acid is at least about 10 nucleotides; preferably at least about 15 nucleotides; and more preferably the length is at least about 20 nucleotides.

In a specific embodiment, the term “standard hybridization conditions” refers to a Tₘ of 55° C., and utilizes conditions as set forth above. In a preferred embodiment, the Tₘ is 60° C.; in a more preferred embodiment, the Tₘ is 65° C. In a specific embodiment, “high stringency” refers to hybridization and/or washing conditions at 68° C. in 0.2×SSC, at 42° C. in 50% formamide, 4×SSC, or under conditions that afford levels of hybridization equivalent to those observed under either of these two conditions.

Suitable hybridization conditions for oligonucleotides (e.g., for oligonucleotide probes or primers) are typically somewhat different than for full-length nucleic acids (e.g., full-length cDNA), because of the oligonucleotides' lower melting temperature. Because the melting temperature of oligonucleotides will depend on the length of the oligonucleotide sequences involved, suitable hybridization temperatures will vary depending upon the oligoncucleotide molecules used. Exemplary temperatures may be 37° C. (for 14-base oligonucleotides), 48° C. (for 17-base oligoncucleotides), 55° C. (for 20-base oligonucleotides) and 60° C. (for 23-base oligonucleotides). Exemplary suitable hybridization conditions for oligonucleotides include washing in 6×SSC/0.05% sodium pyrophosphate, or other conditions that afford equivalent levels of hybridization.

Chaperone Proteins

Chaperonins are a type of molecular chaperone that assist in the folding of nascent proteins in a cell. Chaperonins include both Type I and Type II chaperonins. Type I chaperonins, found in bacteria, include the GroES/GroEL complex found in E. coli. Type II chaperonins are found in the eukaryotic cytosol.

As set forth in the examples provided herein, the present disclosure details experiments that have been performed demonstrating that co-expression of one or more GroESL proteins from E. coli or S. lividans with a gene of interest or nucleotide sequence of interest leads to increased protein yield (e.g., about 5%, 10%, 25%, 50%, 75%, 100%, 150% or more total protein by weight as compared to without co-expression of a chaperone such as GroESL (e.g., a control)). Increased protein yield relative to control can also be about two-fold, three-fold, four-fold, five-fold or more. Experiments of cloning and expressing the biosynthetic gene from the ramoplanin A2 antibiotic producer Actinoplanes showed limited success, that is, co-expression of the gene(s) of interest on a plasmid with a plasmid containing the E. coli GroESL gene led to an increase in soluble protein. Previous experiments had suggested that expression of these genes of interest in a Streptomyces lividans expression system, which exhibited a similar genetic architecture to the native producer, would be successful. Therefore, these genes were cloned into expression plasmids for S. lividans and expressed. Although the proteins produced by the genes of interest were expressed in soluble form, the lack of an inducible system to produce high yield amounts of protein limited this approach. Based on this success, it was decided to attempt to clone the GroESL analog from S. lividans and co-express it on a separate plasmid in an E. coli system. GroES was hypothesized to form the “lid” to the protein folding barrel while GroEL1 and GroEL2 fixated the bulk of the barrel. The reason for the duplication of the GroEL gene was not understood and the first gene, which possessed a high homology to the E. coli GroEL and has been found to exhibit temporal control at room temperature, was selected to be cloned into the plasmid. The GroES-GroEL1 operon was cloned into the pLAC1 plasmid and co-expressed with the plasmids containing the ramoplanin biosynthetic genes. Although a few proteins were not expressed, a majority of the proteins of the ramoplanin cluster were obtained thought is system. Both solubility and total yield was increased in this system as well. In order to determine the broad applicability of this approach to other enzymes, genes which had been previously unsuccessful from other natural product biosynthetic clusters were solicited. Here as well, success was demonstrated with biosynthetic enzymes which had been previously not able to be isolated.

Sequence of pLacI-GroESEL
gaattccggatgagcattcatcaggcgggcaagaatgtgaataaaggc
cggataaaacttgtgcttatttttctttacggtctttaaaaaggccgt
aatatccagctgaacggtaggttataggtacattgagcaactgactga
aatgcctcaaaatgttctttacgatgccattgggatatatcaaggtgg
tatatccagtgatthtttaccattttagcttccttagctcctgaaaat
ctcgataactcaaaaaatacgcccggtagtgatcttatttcattatgg
tgaaagttggaacctcttaccagggtgcgaaacgtaccggcgttcacc
ggaaaaccccgccgacgcgccgccggcattggcactccgcttgaccga
gtgctaatcgcagtcatagtctcggacctggcactccccactggagag
tgccaactacgcgacgggcaggtccggcacccgcgacgacggatccac
ctggtcgccacctcagacagttaaccccgtgagatctccgaaggggag
gtcggatcgtgacgaccaccaptccaaggttgccatcaagccgctcga
ggaccgcatcgtggtccagcptcgacgccgagcagaccacggcttcgg
gcctggtcattccggacacggccaaggagaagccccaggagggcgtcg
tcctggccgtcggcccgggccgcttcgaggacggcaaccgccttccgc
tcgacgtcagcgtcggcgacgtcgtgctctacagcaagtacggcggca
ccgaggtgaagtacaacggcgaggagtacctcgtcctctcggcccgcg
acgtgctcgcgatcgtcgagaagtagaagtagtacttcgcttcaccga
agcaccttgctttccagctgcgcccctggctcccgcgaccataaaaag
ccgggcgtcgggggcgcagttgccgtataaccccaagatttccggcaa
gaggctcacgctcccatggcgaagatcctgaagttcgacgaggacgcc
cgtcgcgccctcgagcgcggcgtcaacaagctcgccgacaccgtgaag
gtgacgatcggccccaaggcgaacgtcgtcatcgacaagaagttcggc
ccccccaccatcaccaacgacggcgtcaccatcgcccgcgaggtcgag
gtcgaggacccgtacgagaacctcggcgcccagctggtgaaggaggtg
gcgaccaagaccaacgacatcgcgggtgacggcaccaccaccgccacc
gtgctcgcccaggcgctcgtgcgcgagggcctgaagaacgtcgccgcc
ggtgcctccccggcgctgctgaagaagggcatcgacgcggccgtcgcc
gccgtgtcggaagaccttctcgccaccgcccgcccgatcgacgagaag
tccgacatcgccgccgtggccgcgctgtccgcccaggaccagcaggtc
ggcgagctgatcgccgaagcgatggacaaggtcggcaaggacggtgtc
atcaccgtcgaggagtccaacaccttcggtctggagctggacttcacc
gagggcatggccttcgacaagggctaccgtctgcgcctacttcgtacg
gaccaggagcgcatggaggccgtcctcgacgacccgtacatcctgatc
aaccagggcaagatctcctccatcgcggacctgctgccgctgctggag
aaggtcatccaggccaacgcctccaagccgctgctgatcatcgccgag
gacctggagggcgaggcgctctccaccctcgtcgtcaaccagatccgc
ggcaccttcaacgcggtggccgtcaaggcccccggcttcggcgaccgc
cgcaaggcgatgctgcaggacatggccgtcctcaccggcgccacggtc
atctccgaggaggtcggcctcaagctcgaccaggtcggcctcgaggtg
ctcggcaccgcccgccgcatcaccgtcaccaaggacgacaccacgatc
gtcgacggtcccggcaagcgcgacgaggtccaggcccgcatcgcccag
atcaaggccgagatcgagaacacggactccgactgggaccgcgagaag
ctccaggagcgcctcgcgaagctggccggcggcgtgtgcgtgatcaag
gtcggcgccgccaccgaggtggagctgaaggagcgcaagcaccgtctg
gaggacgccatctccgcgacccgcgccgcggtcgaggagggcatcgtc
tccggtggtggctccgcgctggtccacgccgtcaaggtgctcgagggc
aacctcggcaagaccggcgacgaggccaccggtgtcgcggtcgtccgc
cgcgccgccgtcgagccgctgcgctggatcgccgagaaccccggcctg
gagggttacgtcatcacctccaaggtcgccgacctcgacaagggccag
ggcttcaacgccgccaccggcgagtacggcgacctggtcaaggccggc
gtcatcgacccggtgaaggtcacccgctccgccctggagaaccccgcc
tccatcgcctccctcctgctgacgaccgagaccctggtcgtcgagaag
aaggaagaggaagagccggccgccggtggccacagccacctaggccac
tcccactgagcgacacgctgagctgagctgagcgaacggtgcccggtc
ccctgcggggggccgggcaccgttctttccaggtgccggttcccgtgc
ccgtcccgggtcggcgtctgccccaccgggttctgccccaccgggttc
gttgccggggtgccgatcaacgtctcattttcgccaaaagttggccca
gggcttcccggtatcaacagggacaccaggatttatttattctgcgaa
gtgatcttccgtcacaggtatttattcggcgcaaagtgcgtcgggtga
tgctgccaacttactgatttagtgtatgatggtgtttttgaggtgctc
cagtggcttctgtttctatcagctgtccctcctgttcagctactgacg
gggtggtgcgtaacggcaaaagcaccgccggacatcagcgctagcgga
gtgtatactggcttactatgttggcactgatgagggtgtcagtgaagt
gcttcatgtggcaggagaaaaaaggctgcaccggtgcgtcagcagaat
atgtgatacaggatatattccgcttcctcgctcactgactcgctacgc
tcggtcgttcgactgcggcgagcggaaatggcttacgaacggggcgga
gatttcctggaagatgccaggaagatacttttacagggaagtgagagg
gccgcggcaaagccgtttttccataggctccgcccccctgacaagcat
cacgaaatctgacgctcaaatcagtggtggcgaaacccgacaggacta
taaagataccaggcgtttcccctggcggctccctcgtgcgctctcctg
ttcctgcctttcggtttaccggtgtcattccgctgttatggccgcgtt
tgtctcattccacgcctgacactcagttccgggtaggcagttcgctcc
aagctggactgtatgcacgaaccccccgttcagtccgaccgctgcgcc
ttatccggtaactatcgtcttgagtccaacccggaaagacatgcaaaa
gcaccactggcagcagccactggtaattgatttagaggagttagtctt
gaagtcatgcgccggttaaggctaaactgaaaggacaagttttggtga
ctgcgctcctccaagccagttacctcggttcaaagagttggtagctca
gagaaccttcgaaaaaccgccctgcaaggcggttttttcgttttcaga
gcaagagattacgcgcagaccaaaacgatctcaagaagatcatcttat
taatcagataaaatatttctagatttcagtgcaatttatctcttcaaa
tgtagcacctgaagtcagccccatacgatataagttgtaattctcatg
ttagtcatgccccgcgcccaccggaaggagctgactgggttgaaggct
ctcaagggcatcggtcgagatcccggtgcctaatgagtgagctaactt
acattaattgcgttgcgctcactgcccgctttccagtcgggaaacctg
tcgtgccagctgcattaatgaatcggccaacgcgcggggagaggcggt
ttgcgtattgggcgccagggtggtttttcttttcaccagtgagacggg
caacagctgattgcccttcaccgcctggccctgagagagttgcagcaa
gcggtccacgctggtttgccccagcaggcgaaaatcctgtttgatggt
ggttaacggcgggatataacatgagctgtcttcggtatcgtcgtatcc
cactaccgagatgtccgcaccaacgcgcagcccggactcggtaatggc
gcgcattgcgcccagcgccatctgatcgttggcaaccagcatcgcagt
gggaacgatgccctcattcagcatttgcatggtttgttgaaaaccgga
catggcactccagtcgccttcccgttccgctatcggctgaatttgatt
gcgagtgagatatttatgccagccagccagacgcagacgcgccgagac
agaacttaatgggcccgctaacagcgcgatttgctggtgacccaatgc
gaccagatgctccacgcccagtcgcgtaccgtcttcatgggagaaaat
aatactgttgatgggtgtctggtcagagacatcaagaaataacgccgg
aacattagtgcaggcagcttccacagcaatggcatcctggtcatccag
cggatagttaatgatcagcccactgacgcgttgcgcgagaagattgtg
caccgccgctttacaggcttcgacgccgcttcgttctaccatcgacac
caccacgctggcacccagttgatcggcgcgagatttaatcgccgcgac
aatttgcgacggcgcgtgcagggccagactggaggtggcaacgccaat
cagcaacgactgtttgcccgccagttgttgtgccacgcggttgggaat
gtaattcagctccgccatcgccgcttccactttttcccgcgttttcgc
agaaacgtggctggcctggttcaccacgcgggaaacggtctgataaga
gacaccggcatactctgcgacatcgtataacgttactggtttcacatt
caccaccctgaattgactctcttccgggcgctatcatgccataccgcg
aaaggttttgcgccattcgatggtgtccgggatctcgacgctctccct
tatgcgactcctgcattaggaagcagcccagtagtaggttgaggccgt
tgagcaccgccgccgcaaggaatggtgtcgtcgccgcacttatgactg
tcttattatcatgcaactcgtaggacaggtgccggcagcgcccaacag
tcccccggccacggggcctgccaccatacccacgccgaaacaagcgcc
ctgcaccattatgttccggatctgcatcgcaggatgctgctggctacc
ctgtggaacacctacatctgtattaacgaagcgctaaccgtttttatc
aggctctgggaggcagaataaatgatcatatcgtcaattattacctcc
acggggagagcctgagcaaactggcctcaggcatttgagaagcacacg
gtcacactttttccggtagtcaataaaccggtaaaccagcaatagaca
taagcggctatttaacgaccctgccctgaaccgacgaccgggtcgaat
ttgattcgaatttctgccattcatccgcttattatcacttattcaggc
gtagcaccaggcgtttaagggcaccaataactgccttaaaaaaattac
gccccgccctgccactcatcgcagtactgttgtaattcattaagcatt
ctgccgacatggaagccatcacagacggcatgatgaacctgaatcgcc
agcggcatcagcaccttgtcgccttgcgtataatatttgcccatggtg
aaaacgggggcgaagaagttgtccatattggccacgtttaaatcaaaa
ctggtgaaactcacccagggattggctgagacgaaaaacatattctca
ataaaccattagggaaataggccaggttttcaccgtaacacgccacat
cttgcgaatatatgtgtagaaactgccggaaatcgtcgtggtattcac
tccagagcgatgaaaacgtttcagtttgctcatggaaaacggtgtaac
aagggtgaacactatcccatatcaccagctcaccgtctttcattgcca
tacg

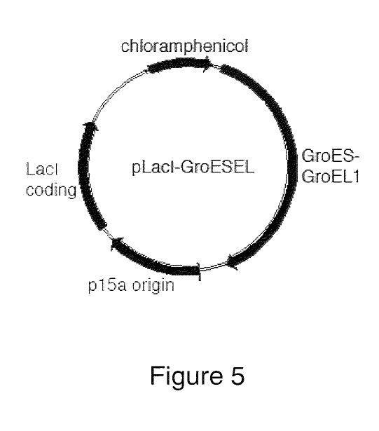

The pLacI plasmid encodes the lac repressor protein (FIG. 5). The p15a origin of replication is compatible with colE1 based vectors, which contain lac operators that regulate T7 promoter-driven expression but do not supply the lac repressor. Co-transformation with pLacI thus supplies a source of lac repressor protein to maintain repression of these vectors in 1DE3 lysogenic bacterial expression host strains. Expression of lad is driven by a constitutive E. coli promoter. The genes groES and groEL may optionally be inserted into the plasmid. Optionally, the gene or nucleotide sequence encoding a protein of interest is inserted to be constitutively expressed.

The methods described herein have many broad implications in the field of molecular biology. The novelty of this method involves the high percentage of success in producing the soluble proteins of interest, which may include antibiotics, anti-cancer compounds such as epothiolone, and the like, as well as the broad applicability of these chaperones to natural product biosynthetic clusters. These methods will enable detailed investigations into substrate specificity, function and mechanism of action of important novel enzymes. Moreover, the information obtained from these methods will enable combinatorial biosynthesis to make, as an example, unique or modified antibiotics and therapeutics at high yield.

Certain aspects of the invention are described in greater detail in the non-limiting Examples that follow.

### EXAMPLES

The studies provided herein have focused on antibiotics for a number of reasons. First, antibiotics are produced by bacteria as a natural defense mechanism. Moreover, it is estimated that over 99% of bacterial natural products are still undiscovered. The genes controlling the production of these natural products are clustered together in the bacterial genome, where genetic sequencing has identified most of the known antibiotic clusters. Lastly, manipulation of these genes would enable different antibiotics to be made on a large scale cost effectively.

### Example 1

Ramoplanin Biosynthetic Proteins: Chaperone proteins have been shown to increase the yield and solubility of proteins expressed in host systems. Previous to this work, the Escherichia coli (E. coli) heat shock proteins, GroES and GroEL, have been characterized and their mechanism of action studied (see, e.g., Buchner, J. et al.). The GroEL and GroES genes encode proteins for 57 kDa and 10 kDa, respectively. GroEL belongs to the Hsp60 family.

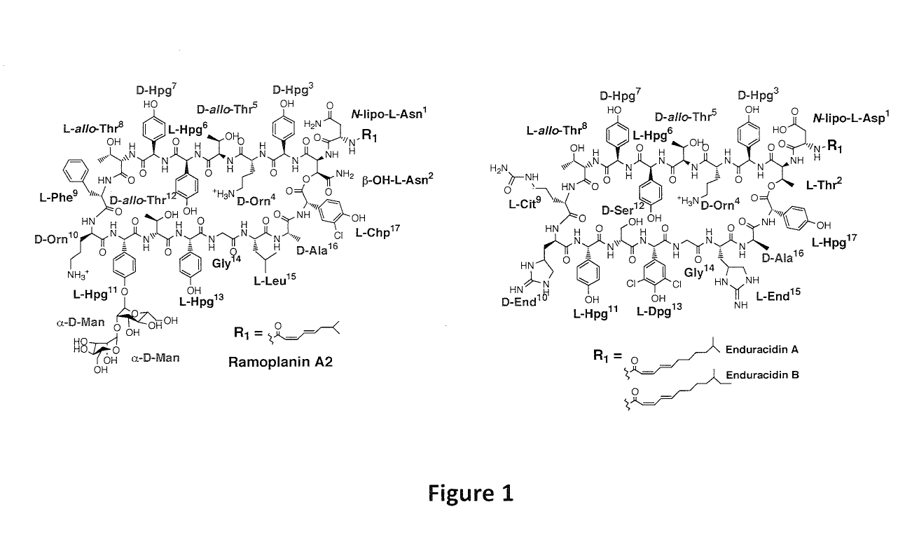

N-acylated antibiotics have demonstrated their importance in treating otherwise resistant infections (Walsh, C. (2003) Antibiotics: Actions, Origins and Resistance, ASM Press, Washington, D.C.). As shown in FIG. 1, ramoplanin A2, a non-ribosomally synthesized peptide antibiotic, is highly effective against several drug-resistant gram-positive bacteria, including vancomycin-resistant Enterococcus faecium (VRE) and methicillin-resistant Staphylococcus aureus (MRSA), two important opportunistic human pathogens (Landman, D. et al. (1996) J. Antimicrob. Chemother. 37:323-329; Romano, G. et al. (1997) J. Antimicrob. Chemother, 39:659-661). Furthermore, ramoplanin does not demonstrate any laboratory or clinical resistance. Recently, the biosynthetic cluster from the ramoplanin producer Actinoplanes (ATCC 33076) was sequenced, revealing an unusual architecture of fatty acid and non-ribosomal peptide synthetase biosynthetic genes (Neu, H. C. et al., (1986) Chemotherapy 32:453-457; Pallanza, R. et al., (1984) J. Antibiot (Tokyo) 37:318-324; Farnet, C. M. et al., (2002) Ramoplanin biosynthesis genes and enzymes of Actinoplanes Appl., P. I., Ed.). Enduracidin, ramoplanin's sister antibiotic, shares a similarly unusual structural architecture (FIG. 1). Interestingly, the N-acyl tail serves as a membrane anchor and incorporates non-proteinogenic amino acids. Understanding how these enzymes cooperatively interact to produce the peptide product will be useful in decoding the molecular logic of non-ribosomal peptide synthetase assembly of complex peptide antibiotics.

A number of non-ribosomal peptide antibiotics contain fatty acyl chains on the amino group of the first acid residue (Walsh, C. supra). The acyl chains on these antibiotics can be straight-chain saturated, terminally branched, or unsaturated. The N-acylation of these antibiotics is likely to function as a membrane anchor to localize products at the membrane interface (Walsh, C., supra). Thorough studies have demonstrated that ramoplanin inhibits peptidoglycan biosynthesis by interfering with the late-stage transglycosylation cross-linking reactions (Somner, E. A. et al., (1990) Antimicrob. Agents Chemother. 34:413-419). Ramoplanin binds to lipid intermediates I and II at different locations than the N-acyl-D-Ala-D-Ala dipeptide site targeted by vancomycin (Fang, X. et al., (2006) Mol. Biosyst. 2:69-76). The fatty acid chain has been demonstrated to be critical for the activity of ramoplanin, although saturation of the double bonds did not dramatically affect its antimicrobial activity (Ciabatti, R. et al., (1992) Hydrogenated derivatives of antibiotic A/16686 [Groupo Lepetit, S. P. A., Ed.] U.S.A.). This fatty acid chain is incorporated into the growing peptide chain by a non-ribosomal peptide synthetase (NRPS). NRPSs are large, multi-functional and multi-modular proteins that selectively bind and activate amino acids before mediating the amino acid condensation into a secondary metabolite (Marahiel, M. A. et al., (1997) Chem. Rev. 97:2651-2673). The minimal NRPS module is composed of two domains, an adenylation domain that binds its cognate amino acid and forms the aminoacyl adenylate, and the thiolation domain, whose long phosphopantetheine arm attaches to the aminoacyl adenylate and transfers it to a downstream domain. A condensation domain mediates the attachment of the amino acyl adenylate waiting on the phosphopantetheine arm and further downstream activated amino acids. Other modifying domains, such as epimerization domains, methylation domains, or cyclization domains can further influence the final secondary metabolite.

NRPS systems can be sub-divided into three types (Finking, R. et al., (2004) Annu. Rev. Microbiol. 58:453-488). Type A NRPSs operate in a co-linear relationship between the modules in the NRPS protein and the amino acids incorporated into the final peptide product. Type B NRPSs function in an iterative manner, repeating the function of each module until the final peptide product is cleaved through cyclization or hydrolysis. Type C NRPSs incorporate qualities of type A and type B NRPSs and often include non-functional modules or domains and lack hypothesized modules or domains.

### Example 2

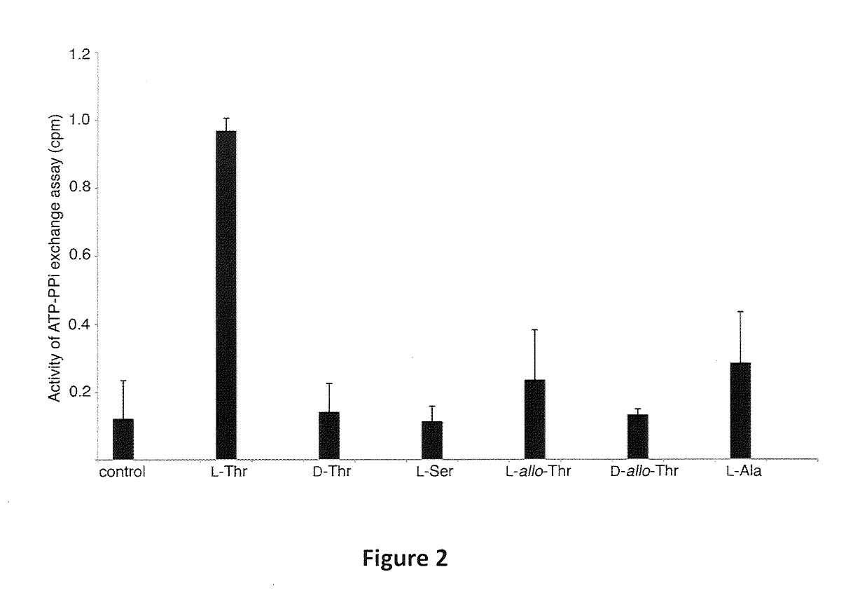

Expression of the Ramoplanin Biosynthetic Proteins: Close inspection of the ramoplanin NRPS indicates that it is a type C NRPS. The NRPS system is composed of six proteins that activate amino acids and condense them to form the final product. The seventeen amino acid secondary metabolite biosynthetic cluster possesses sixteen adenylation domains, indicating that one of the domains must function twice. Ramo12 is hypothesized to activate both the first and second amino acid, L-asparagine, in the final product. Additionally, Ramo12 mediates the attachment of the fatty acid chain to the N-terminus of the growing polypeptide. The Ramo13 NRPS protein is lacking a hypothesized adenylation to activate L-threonine Sequence analysis of the Ramo17 protein reveals an adenylation and thiolation domain which is predicted to activate L-threonine and could function in trans to fulfill this role (Stachelhaus, T. et al., (1999) Chem Biol. 6:493-505). Indeed, as shown in FIG. 2, the ATP-PPi exchange assay utilizing Ramo17 did show activity at L-threonine.

In a similar experiment, ten of the important biosynthetic proteins of ramoplanin (Ramo9, Ramo11, Ramo12, Ramo15, Ramo16, Ramo17, Ramo24, Ramo25, Ramo26 and Ramo27) were highlighted. Several attempts to express these proteins in multiple cell lines, medias, growth conditions, and induction conditions were tried, but unsuccessful. Of the 10, only 3 were successfully obtained, the rest were insoluble or not expressed at all (Table 2). The same genes were co-expressed with GroESL under optimized conditions and the results compared −5 proteins were expressed. Specifically, the E. coli GroESL homolog was obtained from Streptomyces coelicolor. The genes were cloned into a compatible vector for co-expression and evaluated with and without GroESL. As shown in Table 2, 9 of the 10 proteins were isolated, and most saw an increase in soluble protein. These results suggest that for all biosynthetic clusters, organism specific GroESL homologs can increase soluble active protein yield.

### Example 3

Protein images of soluble protein with GroESL: Previously in our laboratory, we cloned and attempted to express the biosynthetic genes from the ramoplanin A2 antibiotic producer Actinoplanes with limited success. Co-expression of our genes of interest on a plasmid with a plasmid containing the E. coli GroESL gene led to an increase in soluble protein. Previous experiments had suggested to us that expression of our genes in a Stretomyces lividans expression system, which exhibited a similar genetic architecture to the native producer, would be successful. Genes were cloned into expression plasmids for S. lividans and expressed. Although the proteins of interest were expressed in soluble form, the lack of an inducible system to produce high yield amounts of protein limited this approach. Based on this success, it was decided to attempt to clone the GroESL analog from S. lividans and co-express it on a separate plasmid in an E. coli system. An analysis of the S. lividans genome revealed three genes comprising the GroESL chaperone system (Betancor et al. Chembiochem 9:2962-6 (2008)). GroES was hypothesized to faun the “lid” to the protein folding barrel while GroEL1 and GreEL2 formed the bulk of the barrel. The reason for the duplication of the GroEL gene was not understood and the first gene, which possessed a high homology to the E. coli GreEL and has been found to exhibit temporal control at room temperature, was selected to be cloned into the plasmid. The GroES-GroEL1 operon was cloned into the pLAC1 plasmid and co-expressed with the plasmids containing the Ramoplanin biosynthetic genes. Although a few proteins were not expressed, a majority of the proteins in the Ramoplanin cluster were obtained through this system. Both solubility and total yield was increased in this system. In order to determine the broad applicability of this approach to other enzymes, genes were solicited which had been previously unsuccessful from other natural products biosynthetic clusters. Here as well, success was demonstrated with biosynthetic enzymes which had been previously not able to be isolated.

Materials and Methods

Materials: Enzymes required for DNA manipulations were from New England Biolabs. Herculase HotStart™ was purchased from Stratagene. The ZeroBlunt™ Cloning kit was purchased from Invitrogen. ³²PPi was purchased from NEN. Media, supplements, and antibiotics were purchased from Sigma and Difco. E. coli competent cells were purchased from Invitrogen and Strategene. Expression plasmids (pET30b) and cloning plasmids (pLAC1) were purchased from Novagen. Contercon concentrators were purchased from Amicon. Desalting columns were purchased from BioRad. All other chemicals were reagent grade and purchased from standard suppliers. The cosmids containing the ramoplanin biosynthetic cluster (008CK, 008Co) were obtained from Ecopia Biosciences. The plasmid containing vbsS, Sfp, and BODIPY-CoA were gifts of Chris Walsh (Harvard Medical School). The plasmid containing pGroESL was a gift from Lortimer (Univ. of Maryland). FPLC purifications were performed on an AKTA FPLC from GE Healthcare. Cell lysis was performed on an Emulsiflex™ emulsifier (Avestin, Ottawa, Ontario, Canada). HPLC purifications were performed on an Agilent 1200 HPLC™. MALDI analysis was performed on an Applied Biosystems Voyager System 6154 instrument. Detailed experimental details can be found in the supplementary materials.

Cloning, expression and purification of Ramoplanin genes: PCR amplification was performed on the cosmid 008CK with the following primers:

SEQ ID NO: 1 - Ramo9F
(5′-GGG AAT TCC ATA TGA GCG CCG CGG GCT CCG
GTT-3′);
SEQ ID NO: 2 - Ramo9R
(5′-CCC AAG CTT GTG GGA GTC GAG GAA CTC GAG
GAT-3′);
SEQ ID NO: 3 - Ramo15R
(5′-CCC AAG CTT GTC ACG GTC CAG GTC GGC GGC
GAT-3′);
SEQ ID NO: 4 - Ramo15F
(5′-GGG AAT TCC ATA TGC AGA AGA TCC CGC TCG
TGT-3′);
SEQ ID NO: 5 - Ramo16F
(5′-GGG AAT TCC ATA TGC GCT TGA CCG GCA AGA
CCC CG-3′);
SEQ ID NO: 6 - Ramo16R
(5′-CCC AAG CTT GCG CGT GGT GAA TCC GCC GTC
GAC-3′);
SEQ ID NO: 7 - Ramo17F
(5′-CAT ACA TAT GCC CAA GTC CCA GCC CGC C-3′);
SEQ ID NO 8 - Ramo17R
(5′-CAT AAA GCT TGG CCG AGC GCA ACG-3′);
SEQ ID NO: 9 - Ramo24F
(5′-GGG AAT TCC ATA TGA CCG CCG CGG CGC TCG
AGA AGC-3′);
SEQ ID NO: 10 - Ramo24R
(5′-CCC AAG CTT GCC GGG GAG CTG ACG GGC GCT
CAG G-3′);
SEQ ID NO: 11 - Ramo25F
(5′-GGG AAT TCC ATA TGA CCG TAC GCC CGC TGG
CGC CAC-3′);
SEQ ID NO: 12 - Ramo25R
(5′-CCC AAG CTT CCG GCC GTC CTC CGC CCG GAC
GGT G-3′);
SEQ ID NO: 13 - Ramo26F
(5′-GGG AAT TCC ATA TGG TCA TCG ACG CCG CCA
CCC AAC-3′);
SEQ ID NO: 14 - Ramo26R
(5′-CCC AAG CTT TCG GCC CGC GCC CGC CTG CAC
CGG C-3′);
SEQ ID NO: 15 - Ramo27F
(5′-GGG AAT TCC ATA TGC CCA ATC CGT TTG AAG
ATC CCG-3′);
SEQ ID NO: 16 - Ramo27R
(5′-CCC AAG CTT GCT CTG CGG TTG CTT CTG CTT
CTC C-3′).

The PCR amplification was optimized for each gene. The typical reaction conditions and cycle consisted of 98° C. for 5 min, 98° C. for 45 sec, 57° C. for 45 sec, 72° C. for 6 min (the last three temp cycles are repeated 30 times), and 72° C. for 10 MIN The PCR mixture consisted of Herculase HotStart™ polymerase, supplied buffer, dNTP mix, primers, 9% DMSO and cosmid DNA.

PCR products were gel purified and ligated into the Zero Blunt™ cloning vector. Successful ligations were sequenced and constructs containing the correct gene sequence were excised and ligated into pET30b with NdeI and HindIII restriction sites. The pET30b constructs containing the gene of interest were transformed into BL21 (DE3) cells at 23° C. The cultures were induced with 100 μM IPTG when the optical density at 600 nm reached 0.6 and allowed to grow overnight. The cells were centrifuged (5K for 10 min) and resuspended in buffer A (50 mM Tris-HCl, pH=8.0, 300 mM NaCl, 10 mM imidazole). The cells were lysed by multiple passages through the Emulsflex™ emulsifier at 10,000 to 15,000 psi. The slurry was centrifuged at 17K rpm for 45 minutes and loaded onto a pre-equilibrated nickel chelating column. The column was washed with 250 ml of buffer A and then subjected to a linear gradient to 100% buffer B (50 mM Tris-HCl, pH=8.0, 300 mM NaCl, 500 mM imidazole). Fractions exhibiting an absorbance at 280 nm were analyzed by SDS-PAGE and pooled. Extinction coefficients were calculated and used to obtain the total yield of the growth. Co-transformations involving pGroESL (exhibiting ampicillin resistance) and pLAC1-GroESL (chloroamphenicol resistance) were purified similarly with the additional antibiotic present during growth.

Cloning of GroESEL1: Genomic DNA from Streptomyces lividans was prepared as previously described (see, e.g., Hopwood). The PCR mixture and conditions were similar to the previously described reaction. The primers were:

SEQ ID NO: 17 -
GroESELF 5′GCA CCC GCG ACG ACG GAT CCA C-3′;
and
SEQ ID NO: 18 -
GroESELR 5′-TCA GTG GGA GTG GCC TAG GTG GCT GTG-3′.

PCR products were ligated into the Zero Blunt™ Cloning vector and sequenced. Once confirmed by sequencing, the insert was excised by digestion with EcoR1 and blunted with Klenow fragment (DNA polymerase I). The pLAC1 vector was digested with BsciA1 and dephosphorylation with Antarctic Phosphatase. The vector and insert were ligated and transformed into DH5□ cells. Transformants were screened for insert and orientation by restriction digest.

ATP-PPi exchange assay: Purified Ramo17 with a C-terminal His₆-tag was concentrated with a 30 kDa Centercon to 3 mLs. The protein was loaded onto a pre-equilibrated desalting column and eluted with 4 mLs of buffer C (50 mM Tris, pH=8.0, 50 mM NaCl). The protein was loaded onto a pre-equilibrated Q-sepharose column and washed with 100 ml of buffer C and a linear gradient of 250 mLs to 100% buffer D (50 mM Tris, pH-8.0, 1M NaCl).

BODIPY-CoA Assay: BODIBY, which is short for boron-dipyrromethene, is a class of fluorescent dyes composed of dipyrrimethene complexed with a disubstituted boron atom, typically a BF₂ unit. The IUPAC name for the BODIPY core is 4,4-difluoro-4-bora-3a,4a-diaza-s-indacene. BODIPY dyes are notable for their uniquely small Stokes shift (high, environment-independent quantum yields, often approaching 100% even in water) and sharp excitation and emission peaks contributing to overall brightness. The combination of these qualities makes BODIPY fluorophore an important tool in a variety of imaging applications. The position of the absorption and emission bands remain almost unchanged in solvents of different polarity as the dipole moment and transition dipole are orthogonal to each other.

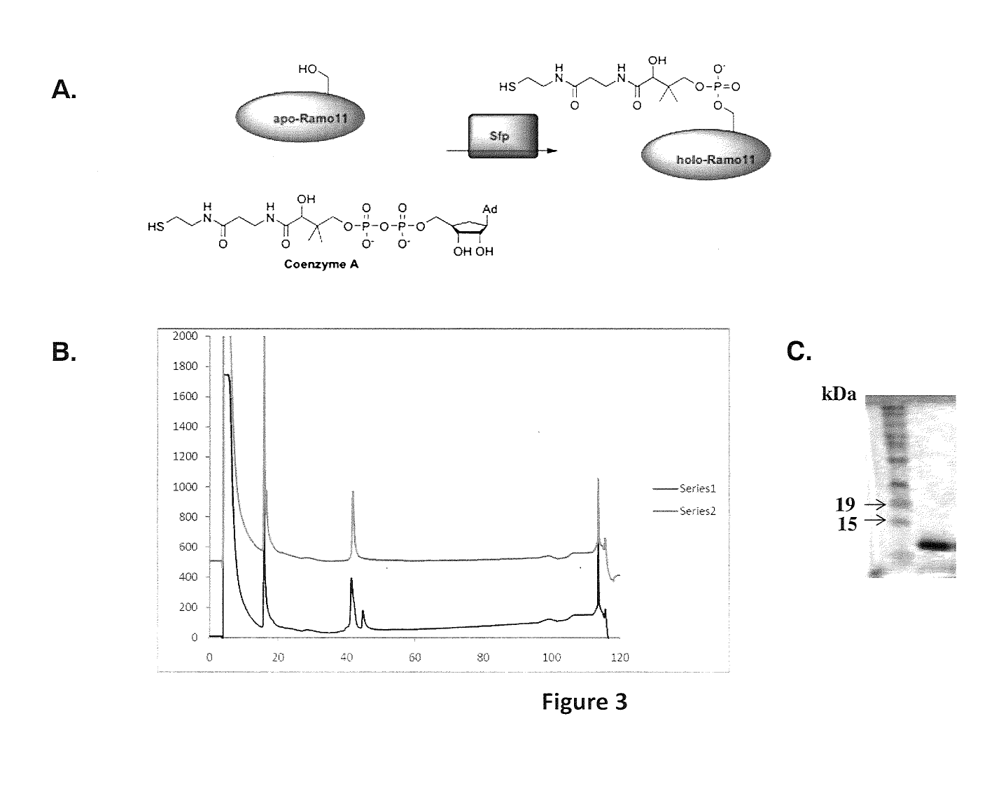

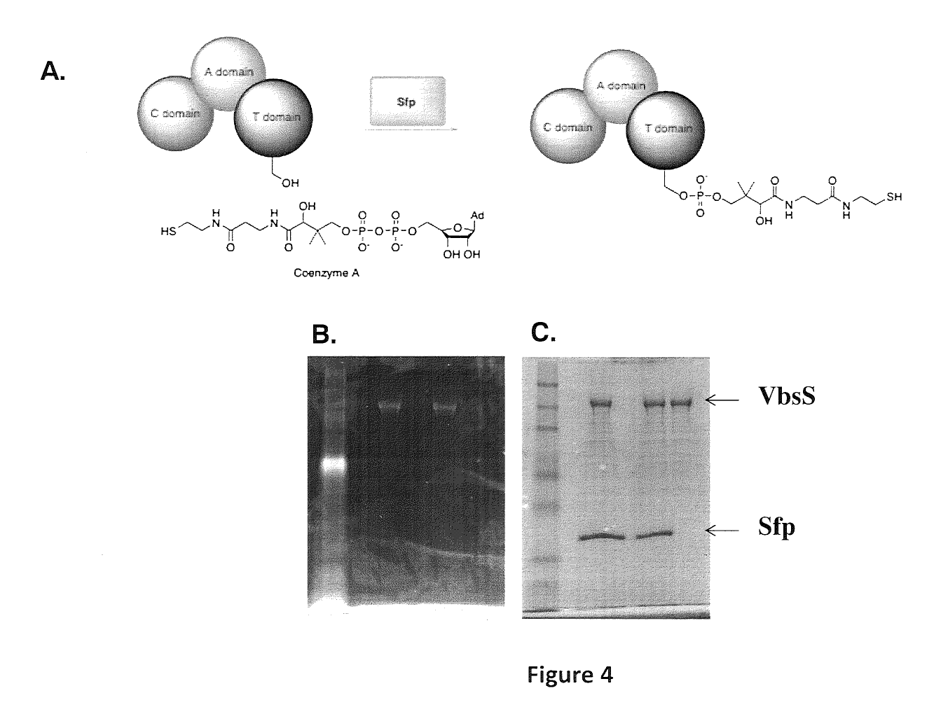

Analysis of the Ramo11 and VbsS clones were analyzed by BODIPY-CoA assays (see FIGS. 3A-C and FIGS. 4A-C, respectively). Specifically, Ramo11 and VbsS clones were incubated with 200 μM BODIPY-CoA and 10 μM Sfp at 37° C. for 1 hr. Reactions were stopped with the addition of 10 mM DTT and 20 μl of 2× loading buffer. Samples were run on 4%-12% SDS-PAGE gels and visualized with UV.

### Example 4

Increased functional expression of the biosynthetic enzymes responsible for the synthesis of Ramoplanin A2 using Streptomyces chaperones. N-acylated antibiotics have demonstrated their importance in treating otherwise resistant infections'. Ramoplanin (FIG. 1), a non-ribosomally synthesized peptide antibiotic, is highly effective against several drug-resistant gram-positive bacteria, including vancomycin-resistant Enterococcus faecium (VRE)² and methicillin-resistant Staphylococcus aureus (MRSA)³, two important opportunistic human pathogens. Recently, the biosynthetic cluster from the ramoplanin producer Actinoplanes ATCC 33076 was sequenced⁴, revealing an unusual architecture of fatty acid and non-ribosomal peptide synthetase biosynthetic genes. Enduracidin, ramoplanin's sister antibiotic, also shares a similar unusual structure. The first step in understanding how these enzymes cooperatively interact to produce the peptide product is expression and isolation of each enzyme to probe its specificity and function. To this end, we have developed a new chaperone expression system to aid in soluble active expression of these and related enzymes.

A large hurdle in the ability to understand the mechanism, function, and substrate specificity of non-ribosomal peptide synthetases (NRPSs) and their tailoring enzymes has been the inability to heterologously express these enzymes. An established and well-studied strategy to increase solubility, co-expression with the chaperonins from Escherichia coli, has been found to be helpful with some enzymes⁵.

Considering the success of a subset of these enzymes' expression in hosts such as Streptomyces coelicolor and S. lividans, it was hypothesized that co-expression of the chaperonins from these organisms in E. coli would provide more soluble active protein with the convenience of established E. coli growth conditions. S. lividans has been used to successfully express prokaryotic and eukaryotic proteins⁶. S. lividans possess two GroEL homologs, GroEL1 and GroEL2, and one GroES⁷. The specific reason for the redundant GroEL protein is not known, although experimental evidence suggests that GroEL1 is under temporal control at 30° C. and GroEL2 is dominant under heat shock conditions⁷. Comparison of the GroES/ GroEL of E. coli to S. lividans GroES/GroEL1/GroEL2 shows a sequence identity of 45, 58%, and 60%, respectively. These differences could possibly explain the different abilities of these chaperonins.

A number of non-ribosomal peptide antibiotics contain fatty acyl chains on the amino group of the first amino acid residue'. The acyl chains on these antibiotics can be straight-chain saturated, terminally branched, or unsaturated. The N-acylation of these antibiotics is likely to function as a membrane anchor to localize products at the membrane interface¹. Thorough studies have demonstrated that ramoplanin inhibits peptidoglycan biosynthesis by interfering with the late-stage transglycosylation cross-linking reactions⁸. Ramoplanin binds to lipid intermediates I and II at different locations than the N-acyl-D-Ala-D-Ala dipeptide site targeted by vancomycin⁹. The fatty acid chain has been demonstrated to be critical for the activity of ramoplanin, although saturation of the double bonds did not dramatically affect its antimicrobial activity¹⁰. This fatty acid chain is incorporated into the growing peptide chain by a non-ribosomal peptide synthetase (NRPS). NRPSs are large multi-functional and multi-modular proteins that selectively bind and activate amino acids before mediating the amino acid condensation into a secondary metabolite¹¹. The minimal NRPS module is composed of two domains, an adenylation domain that binds its cognate amino acid and forms the aminoacyl adenylate, and the thiolation domain, whose long phosphopantetheine arm attaches to the aminoacyl adenylate and transfers it to a downstream domain. A condensation domain mediates the attachment of the amino acyl adenylate waiting on the phosphopantetheine arm and further downstream activated amino acids. Other modifying domains, such as epimerization domains, methylation domains, or cyclization domains can further influence the final secondary metabolite.

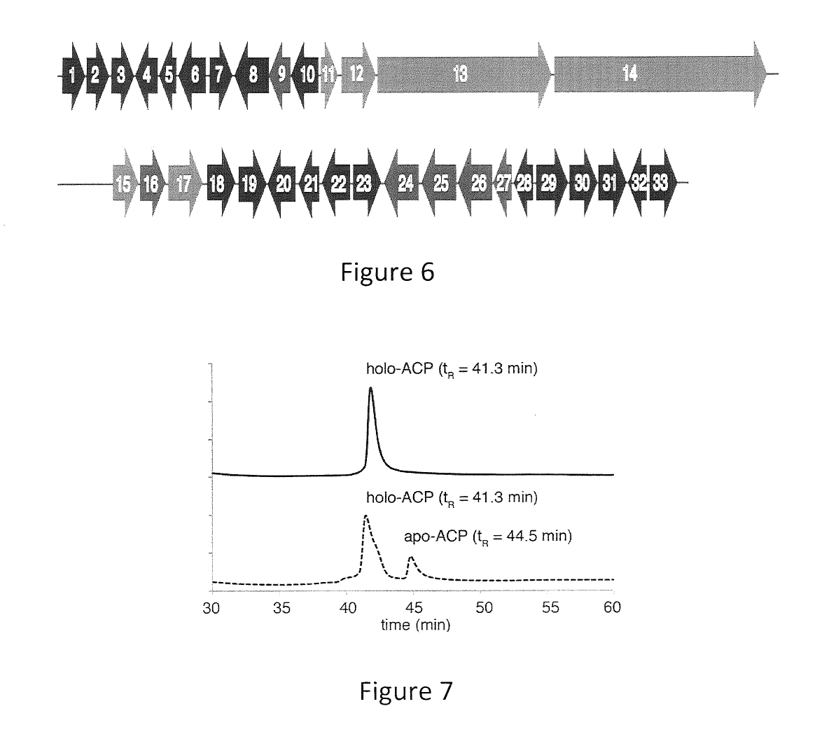

The NRPS system that produces Ramoplanin is composed of six proteins that activate amino acids and condense them to form the final product (FIG. 6). This secondary metabolite is composed of seventeen amino acids; however, the biosynthetic cluster possesses sixteen adenylation domains, indicating that one of the domains must function twice. Ramo12 is hypothesized to activate both the first and second amino acid, L-asparagine, in the final product. Additionally, Ramo12 mediates the attachment of the fatty acid chain to the N-terminus of the growing polypeptide. The Ramo13 NRPS protein is lacking a hypothesized adenylation domain to activate L-threonine. Sequence analysis of the Ramo17 protein reveals an adenylation and thiolation domain which is predicted to activate L-threonine¹² and could function in trans to fulfill this role.

Although required for antibiotic activity, the composition of the N-acyl fatty acid tail was varied without a dramatic effect on the antimicrobial activity. The fatty acid tail attached to Ramoplanin is three carbons shorter than Enduracidin; however, the branching and desaturation is consistent between the two antibiotics. The biosynthesis of this fatty acid and its attachment to the N-terminus of Ramoplanin has been hypothesized¹³. Ramo26 shows high homology to the acyl ligase family of enzymes. This protein potentially ligates the fatty acid chain to Coenzyme A or Ramo11, the acyl carrier protein, for further manipulations of length and saturation of the nascent chain. Blast analysis of Ramo24 and Ramo 25 indicate that both are FAD-dependent dehydrogenases that introduce an α,β desaturation in the fatty acid. Ramo16, a NAD-dependent reductase, mediates the reduction of the carbonyl. Finally, Ramo9 and Ramo15 both have high homology to type II thioesterases, possibly releasing the product to be incorporated into the antibiotic.

Plasmids were constructed to produce C-terminally His₆-tagged expression vectors of genes encoding selected Ramoplanin biosynthetic enzymes. These plasmids were transformed alone and in combination with the E. coli groES/groEL plasmid and the S. lividans groES/groEL1 plasmid into K coli expression cells. The cells were grown to the same optical density during identical growth and induction conditions. The cell pellet was purified by nickel chelating affinity chromotography and fractions containing the protein of interest as determined by SDS-PAGE analysis were pooled and the protein concentration determined. A chart indicating the relative amounts of soluble purified protein is shown in Tables 3 and 4. Co-expression with the S. lividans groES/groEL1 plasmid dramatically increased protein expression levels and in some cases, produced soluble protein for the first time. As a complement to examining the effects of chaperones on protein expression, the use of plasmids encoding rare tRNAs also helped to increase the amount of soluble protein.

In addition to increasing the amount of soluble protein expressed in E. coli, enzymes produced while under the influence of GroES/GroEL1 have exhibited the correct fold and are post-translationally modified. Ramo11, an acyl carrier protein, is hypothesized to shuttle the growing fatty acid chain until it is finally incorporated into the N-terminus of Ramoplanin. In order to perform this task the enzyme must be converted from its apo-form to the holo-form through attachement of the phosphopantetheinyl arm donated by Coenzyme A. If folded correctly, incubation with Sfp, the phosphopantetheinyl transferase from Bacillus subtilis¹⁴, should convert the enzyme from a mixture of apo- and holo-form enzyme to completely holo-form. Ramo11 was purified and incubated with Sfp and Coenzyme A. The reaction was analyzed by HPLC and MALDI analysis to confirm identity (FIG. 7). Ramo11 was successfully completely converted to the active holo-form.

Ramo17, a 95 kDa protein, is an unusual NRPS encoding an external domain of unknown function, an adenylation domain hypothesized to activate L-allo-threonine, and a thiolation domain. Ramo17 is only expressed in the presence of the S. lividans GroES/GroEL1 chaperones. The protein was assayed for folding and activity in two ways. To determine if the protein was folded correctly, Ramo17 was incubated with Sfp, the promiscuous phosphopantetheinyl transferase from Bacillus subtilis¹⁴ and BODIPY-CoA, a fluorescently labeled Coenzyme A analog¹⁵. When analyzed by denaturing polyacrylamide gel electrophoresis and illuminated under fluorescent light, the band corresponding to Ramo17 exhibited fluoresence. This indicates that the CoA analog was covalently attached to the thiolation domain of Ramo17. Second, the specificity of the adenlyation domain was probed with an ATP-PPi exchange assay. Ramo17 is responsible for the addition of the eighth residue in Ramoplanin, L-allo-threonine Since there is not enough data from similar adenylation domain pockets, it is impossible to determine which amino acid, L-allo-threonine or L-threonine, is the preferred substrate based on sequence analysis alone^{12,16}. A panel of amino acids indicate that Ramo17 discriminates between L-allo-threonine, L-threonine, D-allo-threonine, and D-threonine to selectively active only L-threonine (FIG. 2). It is unknown how this transformation between L-threonine and L-allo-threonine is accomplished.

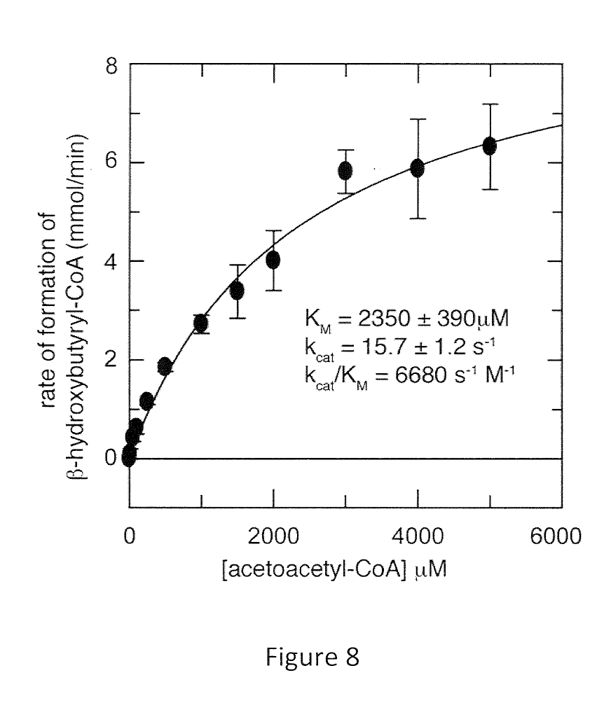

Ramo16, the NAD dependent reductase, was expressed natively in autoinduction media to prevent the formation of insoluble aggregates. Expression in a His₆-tagged expression vector led to protein instability and ultimately precipitation. Ramo16 was assayed for activity in a continuous spectrophotometric assay in the presence of NADH and a pseudo-substrate acetoacetyl-Coenzyme A. The enzyme demonstrated a K_{M} of 2350±390 μM and a k_{cat} of 15.7±1.2 s⁻¹ with 250 μM NADH (FIG. 8).

Others recently demonstrated similar findings while investigating the S. coelicolor chaperonins GroES/GroEL1/GroEL2¹⁷. Sequence comparison of these chaperones reported with chaperones analyzed as described herein show a 97% sequence identity. They adopted a similar approach, however, chose to use the polyketide synthase (PKS) system DEBS 3 from Saccharopolyspora erythraea in their study. Data in their study centers on an already well expressed protein in E. coli expression systems and instead focuses on an increase in catalytic activity.

That reference in combination with this work demonstrates that the chaperones can be used on a variety of systems (NRPS, PKS, fatty acid biosynthesis) from multiple organisms and increase solubility as well as catalytic activity.

The GroES/GroEL chaperonins and their effect on protein folding has been extensively studied^{5b}. Although it appears that many of these large NRPSs are too large to fit in the predicted cavity of the GroEL barrel^{5b}, it may be possible to explain this folding assistance by an alternate model. The GroEL barrel may function without GroES, its 10 kDa subunit that forms the cap on the barrel during protein folding. This mechanism is not unprecedented, prior experiments have found a protein that requires both GroES and GroEL for proper folding¹⁸ and yet is too large to fit in the binding pocket. An alternate mechanism of folding was proposed to explain this anomaly¹⁹. If the GroEL barrel functions without the GroES cap sealing the cavity, then it is possible that each domain or portion of a domain is assisted in its localized folding and is then released. This would allow the GroEL barrel to function repetitiously to achieve the desired result.

Utilization of the chaperones from S. lividans in an E. coli expression system enables the isolation and characterization of proteins from secondary metabolite biosynthetic clusters that have been insurmountable in the past. The first step in understanding the substrate specificity and kinetic parameters of these enzymes is to isolate active soluble protein. This expression system is a significant advance to further the knowledge base of these biosynthetic clusters.

### Experimental Section

Materials: Enzymes required for DNA manipulations were from New England Biolabs. Herculase HotStart was purchased from Stratagene. The ZeroBlunt Cloning kit was purchased from Invitrogen. ³²PPi was acquired from NEN. Media, supplements, and antibiotics were purchased from Sigma and Difco. E. coli competent cells were obtained from Invitrogen and Stratagene. Expression plasmids (pET30b, pET16b) and cloning plasmids (pLacI) were purchased from Novagen. Centricon concentrators were acquired from Amicon. 10DG disposable desalting columns were purchased from BioRad. The Bradford reagents were purchased from Pierce. All other chemicals were reagent grade and purchased from standard suppliers. The cosmids containing the ramoplanin biosynthetic cluster (008CK, 008CO) were obtained from Ecopia Biosciences. The plasmid pGroESL was a gift from Lortimer (Univ. of MD). BODIPY-CoA and Sfp were gifts of Christopher Walsh (Harvard). FPLC purifications were performed on an AKTA FPLC from GE Healthcare. Cell lysis was performed on an Avestin Emulsiflex C-5 homogenizer. HPLC purifications were performed on an Agilent 1200 HPLC. Centrifugation was performed on a Beckman Coulter Optima LE-80K Ultra centrifuge. Absorbance measurements were obtained on a HP 8453 UV-visible spectrophotometer. Scintillation counting was performed by a Wallac 1209 Rack Beta. MALDI analysis was performed on an Applied Biosystems Voyager System 6154 instrument.

Cloning, expression, and purification of Ramo9, Ramo11, Ramo12, Ramo15, Ramo17, Ramo24, Ramo25, Ramo26, and Ramo27: PCR amplification was performed on the cosmid 008CK with the following primers: Ramo9F (5′-GGG AAT TCC ATA TGA GCG CCG CGG GCT CCG GTT-3′) Ramo9R (5′-CCC AAG CTT GTG GGA GTC GAG GAA CTC GAG GAT-3′), Ramo15R (5′-CCC AAG CTT GTC ACG GTC CAG GTC GGC GGC GAT-3′), Ramo15F (5′-GGG AAT TCC ATA TGC AGA AGA TCC CGC TCG TGT-3′), Ramo17F 5′-CAT ACA TAT GCC CAA GTC CCA GCC CGC C-3′, Ramo17R (5′-CAT AAA GCT TGG CCG AGC GCA ACG C-3′), Ramo24F (5′-GGG AAT TCC ATA TGA CCG CCG CGG CGC TCG AGA AGC-3′), Ramo24R (5′-CCC AAG CTT GCC GGG GAG CTG ACG GGC GCT CAG G-3′), Ramo25F (5′-GGG AAT TCC ATA TGA CCG TAC GCC CGC TGG CGC CAC-3′), Ramo25R (5′-CCC AAG CTT CCG GCC GTC CTC CGC CCG GAC GGT G-3′), Ramo26F (5′-GGG AAT TCC ATA TGG TCA TCG ACG CCG CCA CCC AAC-3′), Ramo26R (5′-CCC AAG CTT TCG GCC CGC GCC CGC CTG CAC CGG C-3′), Ramo27F (5′-GGG AAT TCC ATA TGC CCA ATC CGT TTG AAG ATC CCG-3′), Ramo27R (5′-CCC AAG CTT GCT CTG CGG TTG CTT CTG CTT CTC C-3′), Ramo11F, Ramo11R. The PCR amplification was optimized for each gene. The typical reaction conditions and cycle consisted of 98° C. for 5 min, 98° C. for 45 sec, 57° C. for 45 sec, 72° C. for 6 min,(last three temp cycles repeated 30×), and 72° C. for 10 min. PCR mixture consisted of Herculase Hot-Start polymerase, supplied buffer, dNTP mix, primers, 9% DMSO, and cosmid DNA.

PCR products were gel purified and ligated into the Zero Blunt Cloning vector. Successful ligations were sequenced and constructs containing the correct gene sequence were excised and ligated into pET30b with NdeI and HindIII restriction sites. The pET30b constructs containing the gene of interest were transformed into BL21 (DE3) cells at 23° C. The cultures were induced with 100 μM IPTG when the optical density at 600 nm reached 0.6 and allowed to grow overnight. Cells were pelleted and frozen at −20° C. until needed. The cells were centrifuged (5K rpm for 10 min) and resuspended in buffer A (50 mM Tris-HCl, pH=8.0, 300 mM NaCl, 10 mM imidazole). The cells were lysed by multiple passages through the Emulsiflex. The slurry was centrifuged at 40K rpm for 45 minutes and loaded onto a pre-equilibrated nickel-chelating column. The column was washed with 250 mL of buffer A and then subjected to a linear gradient to 100% buffer B (50 mM Tris-HCl, pH=8.0, 300 mM NaCl, 500 mM imidazole). Fractions exhibiting an absorbance at 280 nm were analyzed by SDS-PAGE and pooled. Selected proteins were excised from the acrylamide gel and subjected to trypsin digest and analysis by Q-TOF to confirm identity. A Bradford assay, based on a standard BSA curve, was used to obtain the total yield of the growth. Co-transformations involving pGroESL (exhibiting ampicillin resistance) and pLacI-GroESEL (chloramphenicol resistance) were purified similarly with the additional antibiotic present during growth.

Cloning of GroESEL1: Genomic DNA from Streptomyces lividans was prepared as previously described^{6a}. The PCR mixture and conditions were similar to the previously described reaction. The primers were GroESELF 5′-GCA CCC GCG ACG ACG GAT CCA C-3′, GroESELR 5′-TCA GTG GGA GTG GCC TAG GTG GCT GTG-3′. PCR products were ligated into the Zero Blunt Cloning vector and sequenced. Once confirmed by sequencing, the insert was excised by digestion with EcoRI and blunted with Klenow fragment (DNA polymerase I). The pLacI vector was digested with BsaAI and dephosphorylated with Antarctic Phosphatase. The vector and insert were ligated and transformed into DH5α cells. Transformants were screened for insert and orientation by restriction digest.

Cloning and Purification of Ramo16: The ramo16 gene was obtained by PCR amplification from cosmid DNA (OO8CK) from Actinoplanes ATCC 33076 using Herculase Hot Start Polymerase with the following primers: the C-terminal his-tag were Ramo16C-F (5′-GGG AAT TCC ATA TGC GCT TGA CCG GCA AGA CCC CG-3′), Ramo16C-R (5′-CCC AAG CTT GCG CGT GGT GAA TCC GCC GTC GAC-3′, and the N-terminal his-tag were Ramo16N-F (5′-TAT ACC ATG GCT CGC TTG ACC GGC AAG AC-3′). and Ramo16N-R (5′-TAT AGG ATC CTC AGC GCG TGG TGA ATC CG-3′). The amplified gene products were cloned into the Zero Blunt Cloning Vector and sequenced. The ramo16 insert was digested out of the vector with either NdeI and HindIII or NcoI and BamHI for the C-terminal and N-terminal his-tag constructs, respectively. The inserts were ligated into pET30b expression vector to create pET30b-ramo16C and pET30b-ramo16N. Each plasmid was transformed into BL21 (DE3) for expression. pET30b-ramo16N did not yield any soluble protein and pET30b-ramo16C yielded soluble protein, however, the protein was unstable and inactive. The N-terminal portion of pET30b-ramo16C was digested and ligated into the identically cut C-terminal portion of the pET30b-ramo16N to yield pET30b-ramo16-native. This native protein with no fusion tag was sequenced and then transformed into BL21 (DE3) cells for expression. An overnight culture was grown in LB with 50 μg/mL kanamycin, diluted 1:100 into auto-induction media²⁰ and grown at 23° C. for 48 hours. Cells were harvested and suspended in 50 mM Tris pH 8.0 supplemented with 1 mM DTT and HALT protease inhibitors. Lysis was performed with three passages through the Emulsiflex and centrifuged (40K rpm for 45 minutes). Clarified lysate was applied to a Q column and eluted with a 300 mL gradient of 0 to 350 mM NaCl in the above buffer without protease inhibitors. Fractions containing Ramo16 were pooled and concentrated on a 3 kDa Centricon and injected onto a Superdex S-200 column pre-equilibrated with 50 mM Tris pH=8.0, 100 mM NaCl and 1 mM DTT. Fractions were analyzed by SDS-PAGE and the band corresponding to Ramo16 was excised and the identity of the protein confirmed by Q-TOF. Fractions containing Ramo16 were pooled, concentrated and assayed.

Ramo 16 Activity Assay: Initial rates of Ramo16 were determined by monitoring the decrease in absorbance of NADH at 340 nm at 25° C. The 100 μL reactions were monitored in half-area clear bottom Corning plates using a 96 well plate SpectraMax Molecular Devices spectrophotometer. The extinction coefficient for NADH at 340 nm for 100 μL reactions was 3296 M^{-1 21}. Assays were performed in 50 mM HEPES, pH=7.6, 100 mM NaCl, and 10 μM Ramo16. Initial rates were measured over the first 300 seconds. Rates were determined by varying acetoacetyl-CoA (0 to 5 mM) while NADH was held constant (250 μM).

Ramo11 Activity Assay: Purified Ramo11 (40 μM) was incubated for 1 hr with 5 μM purified Sfp in 50 mM HEPES, pH=7.0 buffer with 2 mM MgCl₂ and 100 μM Coenzyme A. The reaction and a control without Sfp were subjected to HPLC analysis on a linear gradient of H₂O with 0.1% TFA to MeOH with 0.1% TFA on an analytical C18 column (vydac C18 4.6×250 mm). Samples collected at expected retention times were subjected to MALDI analysis and showed masses consistent with holo-ACP (theoretical apo-ACP [M+H]+=12,620 Da, theoretical holo-ACP [M+H]+=13,014 Da, experimental holo-ACP [M+H]+=13.010 Da).

ATP-PPi exchange assay: Purified Ramo11 with a C-terminal His₆-tag was concentrated with a 30 kDa Centricon to 3 mLs. The protein was loaded onto a pre-equilibrated desalting column and eluted with 4 mL of 50 mM Tris, pH=8.0, 50 mM NaCl. The protein was incubated for 30 minutes in 50 mM sodium phosphate, pH=7.8, 1 mM ATP, 0.2 μCi/1 mM ³²PPi, 1 mM MgCl₂, 0.2 mM EDTA, and 1 mM amino acid at 25° C. The reaction was quenched in 1% activated charcoal and 3% perchloric acid and bound to a glass fiber filter. The filter was sequentially washed with 0.2 M sodium phosphate, pH=8.0, H₂O, and finally ethanol. The dried filter was placed in a scintillation vial with 5 mL of scintillation fluid and counted. The experiment was performed in triplicate.

BODIPY-CoA Assay¹⁵: Purified Ramo11 (40 μM) was incubated for 1 hr with 5 μM purified Sfp in 50 mM HEPES, pH=7.0 buffer with 2 mM MgCl₂, 100 μM Coenzyme A, and 40 μM BODIPY-CoA. The reaction and a control without Sfp were analyzed by denaturing polyacrylamide gel electrophoresis and imaged on a Kodak Imaging Station. After imaging the gel under UV light, the gel was stained with Coomassie Blue and a photograph taken. The two images can be compared to indicate which bands were fluorescently labeled.

Any patents or publications mentioned in this specification are indicative of the levels of those skilled in the art to which the invention pertains. These patents and publications, as well as any non-patent documents, are incorporated by reference herein to the same extent as if each individual document was specifically and individually indicated to be incorporated by reference.

One skilled in the art will readily appreciate that the present disclosure is well adapted to carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the disclosure. Changes therein and other uses will occur to those skilled in the art which are encompassed within the spirit of the disclosure as defined by the scope of the claims.

### REFERENCES

1. Walsh, C., Antibiotics: Actions, Origins, and Resistance. ASM Press: Washington, D.C., 2003.
2. Landman et al. Treatment of experimental endocarditis caused by multidrug resistant Enterococcus faecium with ramoplanin and penicillin. J Antimicrob Chemother 1996, 37 (2), 323-9.
3. Romano et al. The effect of ramoplanin coating on colonization by Staphylococcus aureus of catheter segments implanted subcutaneously in mice. J Antimicrob Chemother 1997, 39 (5), 659-61.
4. (a) Neu et al. In vitro activity of A-16686, a new glycopeptide. Chemotherapy 1986, 32 (5), 453-7; (b) Pallanza, R.; Berti, M.; Scotti, R.; Randisi, E.; Arioli, V., A-16686, a new antibiotic from Actinoplanes. II. Biological properties. J Antibiot (Tokyo) 1984, 37 (4), 318-24; (c) Farnet, C. M.; Zazopoulos, E.; Staffa, A. Ramoplanin biosynthesis genes and enzymes of Actinoplanes. 2002.
5. (a) Cole, P. A., Chaperone-assisted protein expression. Structure 1996, 4 (3), 239-42; (b) Walter, S.; Buchner, J., Molecular chaperones—cellular machines for protein folding. Angew Chem Int Ed Engl 2002, 41 (7), 1098-113.
6. (a) Hopwood et al. Genetic Manipulation of Streptomyces: a Laboratory Manual. John Innes Foundation: Normiwch, 1985; (b) Katz et al. Cloning and expression of the tyrosinase gene from Streptomyces antibioticus in Streptomyces lividans. J Gen Microbiol 1983, 129 (9), 2703-14; (c) Gilbert et al. Production and secretion of proteins by streptomycetes. Crit Rev Biotechnol 1995, 15 (1), 13-39.
7. de Leon et al. Streptomyces lividans groES, groEL1 and groEL2 genes. Microbiology 1997, 143 (Pt 11), 3563-71.
8. Somner et al. Inhibition of peptidoglycan biosynthesis by ramoplanin. Antimicrob Agents Chemother 1990, 34 (3), 413-9.
9. Fang et al. The mechanism of action of ramoplanin and enduracidin. Mol Biosyst 2006, 2 (1), 69-76.
10. Ciabatti, R.; Cavalleri, B. Hydrogenated derivatives of antibiotic A/16686. 1992.
11. Marahiel et al. Modular peptide synthetases involved in nonribosomal peptide synthesis. Chem Rev 1997, 97 (7), 2651-2673.
12. Stachelhaus et al. The specificity-conferring code of adenylation domains in nonribosomal peptide synthetases. Chem Biol 1999, 6 (8), 493-505.
13. McCafferty et al. Chemistry and biology of the ramoplanin family of peptide antibiotics. Biopolymers 2002, 66 (4), 261-84.
14. (a) Quadri et al. Identification of a Mycobacterium tuberculosis gene cluster encoding the biosynthetic enzymes for assembly of the virulence-conferring siderophore mycobactin. Chem Biol 1998, 5, 631-645; (b) Zhou et al. Genetically encoded short peptide tags for orthogonal protein labeling by Sfp and AcpS phosphopantetheinyl transferases. ACS Chem Biol 2007, 2 (5), 337-46.
15. La Clair et al. Manipulation of carrier proteins in antibiotic biosynthesis. Chem Biol 2004, 11 (2), 195-201.
16. Challis et al. Predictive, structure-based model of amino acid recognition by nonribosomal peptide synthetase adenylation domains. Chem Biol 2000, 7 (3), 211-224.
17. Betancor et al. Improved catalytic activity of a purified multienzyme from a modular polyketide synthase after coexpression with Streptomyces chaperonins in Escherichia coli. Chembiochem 2008, 9 (18), 2962-6.
18. Dubaquie et al. Identification of in vivo substrates of the yeast mitochondrial chaperonins reveals overlapping but non-identical requirement for hsp60 and hsp10. EMBO J. 1998, 17 (20), 5868-76.
19. Chaudhuri et al. GroEL/GroES-mediated folding of a protein too large to be encapsulated. Cell 2001, 107 (2), 235-46.
20. Studier, F. W., Protein production by auto-induction in high-density shaking cultures. Protein Expres Purif 2005, 41 (1), 207-234.
21. Percival, M. D., Continuous spectrophotometric assay amenable to 96-well plate format for prostaglandin E synthase activity. Anal Biochem 2003, 313 (2), 307-310.

TABLE 1

| | | | | w/S. | |
|---|---|---|---|---|---|
| | | w/o | w/E. coli | Lividans | |
| Protein | Cell Line | GroESL | GroESL | GroESEL | |
| Ramo 9 | BL21 (DE3) | none | 1.9 mg/L | 9.8 | mg/L |
| | | detected | | | |
| Ramo 15 | BL21 (DE3) | 31 mg/L | 45 mg/L | 89 | mg/L |
| Ramo 16 | BL21 (DE3) | none | 2.8 mg/L | 21 | mg/L |
| | | detected | | | |
| Ramo 17 | BL21 (DE3) | none | none | | |
| | | detected | detected | | |
| Ramo 24 | BL21 (DE3) | none | none | x < 1 | mg/L |
| | | detected | detected | | |
| Ramo 25 | BL21 (DE3) | none | none | none | |
| | | detected | detected | detected | |
| Ramo 26 | BL21 (DE3) | 4.6 mg/L | 10 mg/L | 81 | mg/L |
| Ramo 27 | BL21 (DE3) | 120 mg/L | 288 mg/L | 316 | mg/L |

| | | | |
|---|---|---|---|
| VbsS | BL21 (DE3) | none | |
| | | detected | |
| Ramo 24 | BL21 (DE3) | none | none |
| | RP | detected | detected |
| Ramo 25 | BL21 (DE3) | none | none |
| | RP | detected | detected |
| Ramo 26 | BL21 (DE3) | 45 mg/L | 112 mg/L |
| | RP | | |

TABLE 2

| | w/o | | w/E. coli | | w/S. lividans | |
|---|---|---|---|---|---|---|
| Protein | GroESL | | GroESL | | GroESEL | |
| Ramo 9 | none | | 2.1 | mg/L * | 7.5 | mg/L * |
| | detected | | | | | |
| Ramo 11 | ++ | | +++ | | ++++ | |
| Ramo 12 | none | | none | | 4.2 | mg/L * |
| | detected | | detected | | | |
| Ramo 15 | 31 | mg/L | 45 | mg/L | 89 | mg/L |
| Ramo 16 | none | | ++ | | ++++ | |
| | detected | | | | | |
| Ramo 17 | none | | none | | 5.6 | mg/L * |
| | detected | | detected | | | |
| Ramo 24 | none | | None | | x < 1 | mg/L * |
| | detected | | detected | | | |
| Ramo 25 | none | | none | | none | |
| | detected | | detected | | detected | |
| Ramo 26 | 4.6 | mg/L | 10 | mg/L | 81 | mg/L |
| Ramo 27 | 120 | mg/L | 288 | mg/L | 316 | mg/L |

TABLE 3
Non-Ribosomal Peptide Synthetase Biosynthetic Proteins

| | | | E. coli | S. lividans | |
|---|---|---|---|---|---|
| | | No | GroESL | GroESEL | |
| | Cell Line | chaperone | plasmid | plasmid | |
| Ramo 11 | BL21(DE3) | 16 mg/L | — | 16.8 | mg/L |
| Ramo 12 | BL21(DE3) | n.d. ^{[a]} | n.d. ^{[a]} | n.d. ^{[a]} | |
| | BL21(DE3) | n.d. ^{[a]} | n.d. ^{[a]} | — | |
| | RP | | | | |
| Ramo 15 | BL21(DE3) | 31 mg/L | 45 mg/L | 89 | mg/L |
| Ramo 17 | BL21(DE3) | n.d. ^{[a]} | n.d. ^{[a]} | 5.6 | mg/L ^{[b]} |
| Ramo 27 | BL21(DE3) | 120 mg/L | 288 mg/L | 315 | mg/L |

^{[a]} none detected.
^{[b]} Enzyme not completely homogeneous

TABLE 4
Fatty Acid Biosynthetic Proteins

| | | | | E. coli | | S. lividans | |
|---|---|---|---|---|---|---|---|
| | | No | | GroESL | | GroESEL | |
| | Cell Line | chaperone | | plasmid | | plasmid | |
| Ramo 9 | BL21(DE3) | n.d. ^{[a]} | | 1.9 | mg/L ^{[b]} | 9.8 | mg/L ^{[b]} |
| Ramo 16 | BL21(DE3) | 3.6 | mg/L | — | | 11.5 | mg/L |
| Ramo 24 | BL21(DE3) | n.d. ^{[a]} | | n.d. ^{[a]} | | x < 1 | mg/L ^{[b]} |
| | BL21(DE3) | n.d. ^{[a]} | | n.d. ^{[a]} | | — | |
| | RP | | | | | | |
| Ramo 25 | BL21(DE3) | n.d. ^{[a]} | | n.d. ^{[a]} | | n.d. ^{[a]} | |
| | BL21(DE3) | n.d. ^{[a]} | | n.d. ^{[a]} | | — | |
| | RP | | | | | | |
| Ramo 26 | BL21(DE3) | 4.6 | mg/L | 10 | mg/L | 81 | mg/L |

| | | | | | |
|---|---|---|---|---|---|
| BL21(DE3) | 45 | mg/L | 112 | mg/L | — |

RP

^{[a]} none detected.
^{[b]} Enzyme not completely homogeneous

We claim:

1. A method for expressing a protein of interest, said method comprising co-expressing a nucleotide sequence encoding said protein of interest with at least one nucleotide sequence encoding a chaperonin protein in an expression system, to thereby express said protein of interest.

2. The method of claim 1, wherein said chaperonin protein is from the same organism as said protein of interest.

3. The method of claim 1, wherein said expression system comprises E. coli.

4. The method of claim 1, wherein said chaperonin protein is a Streptomyces lividans chaperonin protein.

5. The method of claim 1, wherein said chaperonin protein is selected from the group consisting of: GroES, GroEL1 and GroEL2.

6. The method of claim 5, wherein said chaperonin protein is a Streptomyces lividans chaperonin protein.

7. The method of claim 1, in which said protein of interest is encoded by a gene of a biosynthetic cluster.

8. The method of claim 1, in which said protein of interest is an enzyme involved in the biosynthesis of an antibiotic.

9. The method of claim 8, wherein said antibiotic is a lipopeptide antibiotic.

10. The method of claim 8, wherein said antibiotic is selected from the group consisting of surfactin, ramoplanin, daptomycin and mycosubtilin.

11. The method of claim 8, wherein said antibiotic is ramoplanin.

12. The method of claim 1, wherein said nucleotide sequence encoding said protein of interest and said nucleotide sequence encoding said chaperonin protein are on one plasmid.

13. The method of claim 12, wherein said plasmid comprises pLAC1.

14. The method of claim 1, wherein said nucleotide sequence encoding said protein of interest and said nucleotide sequence encoding a chaperonin protein are on different plasmids.

15. A composition comprising an isolated protein of interest, wherein said protein of interest is encoded by a gene of a biosynthetic cluster.

16. The composition of claim 15, wherein said protein of interest is soluble.

17. The composition of claim 15, wherein said protein of interest is an enzyme or portion thereof involved in the biosynthesis of an antibiotic.

18. The composition of claim 15, wherein said protein of interest is a non-ribosomal peptide synthetase (NRPS) or a portion thereof.

19. The composition of claim 15, wherein said protein of interest is an enzyme or portion thereof involved in the biosynthesis of an antibiotic selected from the group consisting of surfactin, ramoplanin, daptomycin and mycosubtilin.

20. The composition of claim 15, wherein said protein of interest is produced by the process of co-expressing a nucleotide sequence encoding said protein of interest with at least one nucleotide sequence encoding a chaperonin protein in an expression system, to thereby express said protein of interest.

21. The composition of claim 20, wherein said chaperonin protein is from the same organism as said protein of interest.

22. The composition of claim 20, wherein said expression system comprises E. coli.

23. The composition of claim 15, wherein said protein of interest is selected from the group consisting of: Ramo 11, Ramo 15, Ramo 17, Ramo 27, Ramo 9, Ramo 16, and Ramo 26.


SEQUENCE LISTING (21 sequences)

SEQ ID NO: 1 - DNA, 33 bp, Artificial
  PCR primer
gggaattccatatgagcgccgcgggctccggtt

SEQ ID NO: 2 - DNA, 33 bp, Artificial
  PCR primer
cccaagcttgtgggagtcgaggaactcgaggat

SEQ ID NO: 3 - DNA, 33 bp, Artificial
  PCR primer
cccaagcttgtcacggtccaggtcggcggcgat

SEQ ID NO: 4 - DNA, 33 bp, Artificial
  PCR primer
gggaattccatatgcagaagatcccgctcgtgt

SEQ ID NO: 5 - DNA, 35 bp, Artificial
  PCR primer
gggaattccatatgcgcttgaccggcaagaccccg

SEQ ID NO: 6 - DNA, 33 bp, Artificial
  PCR primer
cccaagcttgcgcgtggtgaatccgccgtcgac

SEQ ID NO: 7 - DNA, 28 bp, Artificial
  PCR primer
catacatatgcccaagtcccagcccgcc

SEQ ID NO: 8 - DNA, 24 bp, Artificial
  PCR primer
cataaagcttggccgagcgcaacg

SEQ ID NO: 9 - DNA, 36 bp, Artificial
  PCR primer
gggaattccatatgaccgccgcggcgctcgagaagc

SEQ ID NO: 10 - DNA, 34 bp, Artificial
  PCR primer
cccaagcttgccggggagctgacgggcgctcagg

SEQ ID NO: 11 - DNA, 36 bp, Artificial
  PCR primer
gggaattccatatgaccgtacgcccgctggcgccac

SEQ ID NO: 12 - DNA, 34 bp, Artificial
  PCR primer
cccaagcttccggccgtcctccgcccggacggtg

SEQ ID NO: 13 - DNA, 36 bp, Artificial
  PCR primer
gggaattccatatggtcatcgacgccgccacccaac

SEQ ID NO: 14 - DNA, 34 bp, Artificial
  PCR primer
cccaagctttcggcccgcgcccgcctgcaccggc

SEQ ID NO: 15 - DNA, 36 bp, Artificial
  PCR primer
gggaattccatatgcccaatccgtttgaagatcccg

SEQ ID NO: 16 - DNA, 34 bp, Artificial
  PCR primer
cccaagcttgctctgcggttgcttctgcttctcc

SEQ ID NO: 17 - DNA, 22 bp, Artificial
  PCR primer
gcacccgcgacgacggatccac

SEQ ID NO: 18 - DNA, 27 bp, Artificial
  PCR primer
tcagtgggagtggcctaggtggctgtg

SEQ ID NO: 19 - DNA, 29 bp, Artificial
  PCR primer
tataccatggctcgcttgaccggcaagac

SEQ ID NO: 20 - DNA, 29 bp, Artificial
  PCR primer
tataggatcctcagcgcgtggtgaatccg

SEQ ID NO: 21 - DNA, 6253 bp, Artificial
  pLacI-GroESEL plasmid sequence
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttaccagggtgcgaaacgtaccggcgttcaccggaaaaccccgccgacgcgccgccggcattggcactccgcttgaccgagtgctaatcgcagtcatagtctcggacctggcactccccactggagagtgccaactacgcgacgggcaggtccggcacccgcgacgacggatccacctggtcgccacctcagacagttaaccccgtgagatctccgaaggggaggtcggatcgtgacgaccaccagctccaaggttgccatcaagccgctcgaggaccgcatcgtggtccagccgctcgacgccgagcagaccacggcttcgggcctggtcattccggacacggccaaggagaagccccaggagggcgtcgtcctggccgtcggcccgggccgcttcgaggacggcaaccgccttccgctcgacgtcagcgtcggcgacgtcgtgctctacagcaagtacggcggcaccgaggtgaagtacaacggcgaggagtacctcgtcctctcggcccgcgacgtgctcgcgatcgtcgagaagtagaagtagtacttcgcttcaccgaagcaccttgctttccagctgcgcccctggctcccgcgaccataaaaagccgggcgtcgggggcgcagttgccgtataaccccaagatttccggcaagaggctcacgctcccatggcgaagatcctgaagttcgacgaggacgcccgtcgcgccctcgagcgcggcgtcaacaagctcgccgacaccgtgaaggtgacgatcggccccaaggcgaacgtcgtcatcgacaagaagttcggcccccccaccatcaccaacgacggcgtcaccatcgcccgcgaggtcgaggtcgaggacccgtacgagaacctcggcgcccagctggtgaaggaggtggcgaccaagaccaacgacatcgcgggtgacggcaccaccaccgccaccgtgctcgcccaggcgctcgtgcgcgagggcctgaagaacgtcgccgccggtgcctccccggcgctgctgaagaagggcatcgacgcggccgtcgccgccgtgtcggaagaccttctcgccaccgcccgcccgatcgacgagaagtccgacatcgccgccgtggccgcgctgtccgcccaggaccagcaggtcggcgagctgatcgccgaagcgatggacaaggtcggcaaggacggtgtcatcaccgtcgaggagtccaacaccttcggtctggagctggacttcaccgagggcatggccttcgacaagggctaccgtctgcgcctacttcgtacggaccaggagcgcatggaggccgtcctcgacgacccgtacatcctgatcaaccagggcaagatctcctccatcgcggacctgctgccgctgctggagaaggtcatccaggccaacgcctccaagccgctgctgatcatcgccgaggacctggagggcgaggcgctctccaccctcgtcgtcaaccagatccgcggcaccttcaacgcggtggccgtcaaggcccccggcttcggcgaccgccgcaaggcgatgctgcaggacatggccgtcctcaccggcgccacggtcatctccgaggaggtcggcctcaagctcgaccaggtcggcctcgaggtgctcggcaccgcccgccgcatcaccgtcaccaaggacgacaccacgatcgtcgacggtcccggcaagcgcgacgaggtccaggcccgcatcgcccagatcaaggccgagatcgagaacacggactccgactgggaccgcgagaagctccaggagcgcctcgcgaagctggccggcggcgtgtgcgtgatcaaggtcggcgccgccaccgaggtggagctgaaggagcgcaagcaccgtctggaggacgccatctccgcgacccgcgccgcggtcgaggagggcatcgtctccggtggtggctccgcgctggtccacgccgtcaaggtgctcgagggcaacctcggcaagaccggcgacgaggccaccggtgtcgcggtcgtccgccgcgccgccgtcgagccgctgcgctggatcgccgagaaccccggcctggagggttacgtcatcacctccaaggtcgccgacctcgacaagggccagggcttcaacgccgccaccggcgagtacggcgacctggtcaaggccggcgtcatcgacccggtgaaggtcacccgctccgccctggagaaccccgcctccatcgcctccctcctgctgacgaccgagaccctggtcgtcgagaagaaggaagaggaagagccggccgccggtggccacagccacctaggccactcccactgagcgacacgctgagctgagctgagcgaacggtgcccggtcccctgcggggggccgggcaccgttctttccaggtgccggttcccgtgcccgtcccgggtcggcgtctgccccaccgggttctgccccaccgggttcgttgccggggtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgtcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg